(19) United States
(12) Patent Application Publication
(10) Pub. No.: US 2004/0136980 A1
(43) Pub. Date: Jul. 15, 2004

(54) Thrombopoietin(tpo) synthebody for stimulation of platelet production

(76) Inventors: Daniel A Soltis, Belle Mead, NJ (US); Ronald M. Burch, Wilton, CT (US); Robert A. Ogert, Hoboken, NJ (US)

Correspondence Address: DARBY & DARBY P.C., P. O. BOX 5257, NEW YORK, NY, 10150-5257, US

(21) Appl. No.: 10/469,124

(22) PCT Filed: Apr. 2, 2002
(86) PCT No.: PCT/US02/10301

(51) Int. Cl. A61K 39/395; C07K 16/44

(52) U.S. Cl. 424/132.1; 530/387.3

(57) ABSTRACT

The present invention relates to a synthetic variable region of an immunoglobin construct which contains in at least one of its CDRs a sequence of thrombopoietin, <i>e.g.</i>, IEGPTLRQWLAARA or its derivatives. This construct can efficiently bind and activate a thrombopoientin receptor (MPL) leading to stimulation of proliferation, growth or differentiation or modulation of apoptosis of hematopoietic cells, especially platelet progenitor cells. The invention further relates to the use of the synthebody to treat hematopoietic or immune disorders, and particularly thrombocytopenia resulting from chemotherapy, radiation therapy, or bone marrow transfusions.

### FIELD OF THE INVENTION

The present invention relates to constructs, e.g., synthetic antibodies (synthebodies) that stimulate proliferation and/or differentiation and/or modulate apoptosis of hematopoietic cells, especially platelet progenitor cells. Such constructs are capable of binding to and activating a thrombopoietin (TPO) receptor (TPOR/MPL/c-MPL). The invention further relates to the use of these constructs to treat hematopoietic or immune disorders, and particularly thrombocytopenia resulting from chemotherapy, radiation therapy, or hematopoietic progenitor cell ablation in connection with bone marrow transfusions.

### BACKGROUND OF THE INVENTION

Each day an adult human produces 2×10¹¹ red blood cells, and about one-half as many white cells and platelets. The level of each cell type present in blood is normally maintained within a very narrow range; however, in times of increased demand, individual cell production can rise 10-fold or more. It is well established that blood cell generation is subject to a very tight homeostatic control. In humans, nearly all blood cell production occurs in the red bone marrowthat represents a hierarchical developmental system composed of hematopoietic stem cells, intermediate level progenitors and maturing cells committed to each lineage.

Although the morphology of all the major blood cell types is similar through their initial development stages, megakaryocytes, cells committed to platelet production, are marked by an obvious structural and functional departure beyond the blast cell level of differentiation (for review see Kaushansky, BioEssays, 21: 353-360, 1999): growing to a size 10 times the diameter of most other bone marrow and blood cells, and containing up to 128 times the normal chromosomal complement, these cells give rise to blood platelets. After a series of normal cell divisions, the developing megakaryocyte precursor enters a unique cell cycle characterized by a brief (˜1 h) G₁ phase, a typical (7 h) S phase, a very brief (˜45 min) G₂ phase, followed by the endomitotic phase (an aborted M phase) (Jackson, Int. J. Cell Cloning, 8: 224-6, 1990; Debili et al., Keystone Meeting: “Molecular Regulation of Platelet Production,” Incline Village, Nev., Jan. 10-15, 1998). Once the cell develops a highly polyploid nucleus, it also develops demarcation membranes necessary for cytoplasmic fragmentation. This event is accompanied by expression of glycoprotein GPIIbIIIa (platelet fibrinogen receptor; Papayannopoulou et al., Exp. Hematol., 24: 660-9, 1996) and GPIb (von Willibrand factor receptor; Kaushansky et al., Nature, 369: 568-571, 1994), the granules that contain ADP, serotonin, -thromboglobulin, and other substances critical for mature platelet function. Finally, highly polyploid megakaryocytes undergo cytoplasmic partitioning, allowing the release of thousands of platelets (Choi et al., Blood, 85: 402-413, 1995; Cramer et al., Blood, 89: 2336-2346, 1997).

Like all blood cell precursors, megakaryocytes are derived from pluripotent marrow stem cellsthat retain the capacity to extensively self-renew, or to differentiate into all of the elements of the blood. Although mounting evidence indicates that stem cell lineage commitment decisions are cell autonomous (Fairbarim, Cell, 74:823-832, 1993), external influences, exerted primarily by a family of structurally related glycoprotein cytokines and hormones, are required for these programs to become manifest (Ogawa, Blood, 81: 2844-2853, 1993).

In fact, platelet production is primarily regulated by signaling mechanisms induced by interaction between thrombopoietin (TPO) and its cellular receptor TPOR/MPUc-MPL.

### Thrombopoietin (TPO)

Thrombopoietin (TPO) is a key hematopoietic growth factor involved in stimulation of megakaryocytopoiesis and platelet production. TPO is expressed in liver and kidney, and, in response to platelet demand, its expression may be also upregulated in the bone marrow microenvironment (Kato et al., Stem Cells, 16: 322-328, 1998; McCarty et al., Blood, 86:3668-3675, 1995). As TPO expression is mostly constitutive, the TPO levels are believed to be regulated by sequestering by platelets. Indeed, TPO receptors (TPOR/MPL) localized on platelets were shown to bind TPO with high affinity (Kd=100-400 pM) followed by TPO internalization and degradation (Fielder et al., Blood 87: 2154, 1996).

The gene encoding TPO has been cloned and characterized (Kuter et al., Proc. Natl. Acad. Sci. USA, 91:11104-11108, 1994; Bartley et al., Cell, 77:1117-1124, 1994; Kaushansky et al., Nature, 369:568-571, 1994; Wendling et al., Nature, 369:571-574, 1994, and de Sauvage et al., Nature, 369:533-538, 1994). Human TPO (hTPO) cDNA encodes a 353 amino acid-long polypeptide. The full-length hTPO secreted from mammalian cells after cleavage of the signal peptide consists of 332 amino acids. Although the predicted molecular mass of this protein is 38 kD, the molecular masses reported from measurements of material in serum or in culture fluid from recombinant cells vary from 18 to 85 kD, suggesting that TPO is not only highly glycosylated, but also that post-translational proteolytic processing may occur (Kato et al., Stem Cells, 14[suppl.1]: 139-147, 1996; Foster and Lok, Stem Cells, 14[suppl.1]: 102-107, 1996).

There is a high degree of sequence homology (greater than 70%) between human, porcine, canine, murine, and rat TPO as determined according to an alignment scheme such as by the Cluster Method, wherein similarity is based on the MEGALIGN algorithm. Homology is higher in the N-terminal part than in the C-terminal part of the molecule. Indeed, TPO appears to have two distinct regions separated by a conserved Arg-Arg (a potential proteolytic cleavage site).

The N-terminal region of TPO (154 aa residues) has 20% sequence identity, 25% similarity with erythropoietin (EPO) and some homology with interferon- and interferon-. Two disulfide bonds in the N-terminal domain (Cys7-Cys151 and Cys29-Cys85) appear to be essential for the biological activity of TPO (Kato et al., 1996, supra; Foster and Lok, 1996, supra; Wada et al., Biochem. Biophys. Res. Commun., 213:1091-1098, 1995; Hoffman et al., Biochemistry, 35: 14849-61, 1996). Although the crystal or NMR structure of the TPO molecule is not yet available, recent studies indicate that all biologically active domains of TPO (i.e., domains involved in TPO receptor binding and signaling leading to stimulation of megakaryocyte proliferation and differentiation) are contained within its N-terminal conserved portion (Kato et al., 1996, supra; de Sauvage et al., 1994, supra; Harker et al., Blood, 88: 511-521, 1996).

The C-terminal region of TPO shows wide species divergence. This region is highly glycosylated and contains 6N-linked and multiple O-linked carbohydrate chains (Kato et al., Proc. Natl. Acad. Sci. USA, 94: 4669-4674, 1997) and is thought to be necessary for survival of TPO in the circulation. In addition, the sugar chains have been shown to be important for the secretion of TPO from cells (Linden and Kaushansky, Blood, 90[suppl.]:55a, 1997).

TPO is the primary regulator of physiological platelet production. Indeed, at therapeutic doses, it causes as much as a 10-fold increase in circulating platelet levels (Debili et al., supra). The primary target cell population for TPO in bone marrow comprises megakaryocyte progenitors at the late stage of differentiation, such as colony-forming unit-megakaryocyte (CFU-MK) expressing GPIIb/IIIa (CD41) (Miyazaki et al., Exp. Hematol., 20: 855-861, 1992; Miyazaki et al., Exp. Hematol., 23:1224-1228, 1995; Kato et al., Exp. Hematol., 24: 1209-1214, 1996). TPO has a dramatic effect on both proliferation and differentiation of megakaryocytes (in vitro and in vivo) and is the most potent thrombopoietic agent described to date (Lok et al., Nature, 369: 565, 1994; de Sauvage et al., 1994, supra; Bartley et al., 1994, supra; Kuter, Curr. Opin. Hematol., 4: 163, 1997; Kaushansky et al., Nature, 369:568, 1994; Choi et al., 1995, supra; Broudy et al., Blood, 85: 1719, 1995; Zeigler et al., Blood, 84:4045, 1994; Papayannopoulou et al., Blood, 84: 32, 1994; Wendling et al., 1994, supra; Banu et al., Blood, 86: 1331, 1995; Debili et al., Blood, 86: 2516, 1995; Kaushansky et al., Proc. Natl. Acad. Sci. USA, 92: 3234, 1995). In fact, neither accessory cells, nor serum components are required for TPO to induce megakaryocyte growth and differentiation in vitro. Interestingly, however, being essential for full maturation of megakaryocytes (Debili et al., 1995, supra; Kaushansky et al., Proc. Natl. Acad. Sci. USA, 92: 3234-3238, 1995; Zucker-Franklin and Kaushansky, Blood, 88: 1632-1638, 1996), TPO does not exert the direct effect on platelet shedding from mature megakaryocytes (Choi et al., Br. J. Haematol., 95:227-233, 1996; Horie et al., Exp. Hematol., 25:169-176, 1997). Besides stimulating megakaryocyte differentiation and proliferation, TPO also acts in vitro and in vivo to prevent programmed cell death (apoptosis) in both normal megakaryoctyes and their progenitors (Borge et al., Blood, 88:2859-70, 1996; Borge et al., Blood, 90:2282, 1997; Nagasawa et al., Exp. Hematol., 25:897, 1997).

Based on the data presented above, it has been proposed that TPO can be used as a therapeutic for treating various forms of thrombocytopenia, a life-threatening decrease in production of platelets. Indeed, early results from human clinical trails showed that administration of recombinant TPO (rhTPO) stimulates platelet production in humans. In phase I trials, a pegylated and truncated form of recombinant TPO (MGDF) administered daily for 10 days at 0.03-5.0 μg/kg to cancer patients prior to chemotherapy caused up to a 4-fold increase in circulating platelet levels (Basser et al., Blood, 86[suppl. 1]: 257, 1995; Rasko et al., Blood, 86[suppl.1]: 497, 1995). Similarly, patients given a single dose of recombinant TPO had platelet levels increase by 4-fold (Vaden-Raj et al., 1996). In both studies platelet increases were observed by day 4 and reached maximum about 12-16 days later. Similarly, pegylated MGDF given post-chemotherapy to myelosuppressed patients has been shown to reduce the extent of the platelet nadir (Begley et al., Proceedings of ASCO, 15: 271, 1996; Fanucchi et al., Proceedings of ASCO, 15: 271, 1996).

It follows, that TPO or its functional homolog would be particularly useful as a therapeutic for treating thrombocytopenia-associated bone marrow hypoplasia resulting from chemotherapy, radiation therapy or bone marrow transfusion.

Cancer remains the second leading cause of death in the United States. In 1999, more than 1.2 million Americans were diagnosed with cancer (American Cancer Society: Cancer facts and figures). The majority of these cancer victims will receive some form of radiation or chemotherapy treatment that can induce thrombocytopenia. It follows, that development of products to treat thrombocytopenia could help improve the management of 600,000-800,000 cancer patients as well as of a great number of patients undergoing bone marrow transfusions.

As outlined above, even though TPO dramatically stimulates platelet production, it only has a modest effect on platelet function. Indeed, an increase in thrombotic episodes in animals and humans treated with recombinant TPO has never been observed, even when platelet levels were 4-10 fold above normal (Harker et al., Blood, 87: 1833, 1996; Toombs et al., Thromb. Res., 80: 23, 1995; Toombs et al., Blood, 86[suppl.1]: 369, 1995). It follows, that stimulation of platelet production by TPO will unlikely be associated with an increase in thromboocculsive events.

Multiple cytokines (e.g., stem cell factor [SCF], IL-1, IL-3, IL-6, IL-11, leukaemia inhibiting factor [LIF], G-CSF, GM-CSF, M-CSF, erythropoietin (EPO), kit ligand, and -interferon) have been shown to possess thrombocytopoietic activity (for review see Kaushansky et al., Blood, 86:419-431, 1995; Muraoka et al., Br. J. Haematol., 98:265-273, 1997). However, in contrast to TPO, these cytokines have pleiotropic actions and their thrombocytopoietic activity is much weaker than that of TPO. Indeed, very high concentrations of IL-6 or IL-11 in combination with high concentrations of SCF or IL-3 are required to approach the activity of TPO alone. Moreover, with the exception of IL-3, all these cytokines have only a synergistic and additive effect on thrombocytopoiesis, i.e., the presence of TPO is obligatory (Broudy et al., Blood, 85:1719-1726, 1995; Kaushansky et al., Blood, 1995, supra). IL-3 appears to be the only other cytokine with independent megakaryocytopoietic activity. However, even IL-3 can only promote partial megakaryocyte differentiation with TPO still being required for megakaryocyte polyploidization and maturation.

In addition to its role in megakaryocytopoiesis, TPO acts on pluripotent stem cells with long-term culture and re-populating capacity, in synergy with early-acting growth factors such as stem cell factor (SCF or c-kit ligand), flt3 ligand (FL) and IL-3. This effect has been found with cells from different sources, such as bone marrow, peripheral blood and cord vein blood (in humans the CD34⁺CD38⁻ cell subset). Moreover, the whole hematopoietic progenitor cell compartment (granulocytic macrophage, erythroid, megakaryocytic) seems to respond to TPO, both in vitro and in vivo (Fibbe et al., Blood, 86:3308-3313, 1995; Kaushansky et al., J. Clin. Invest., 96:1683-1687, 1995; Borge et al., Blood, 88:2859-2870, 1996; Itoh et al., Br. J. Haematol., 94:228-235, 1996; Kaushansky et al., Exp. Hematol., 24:265-269, 1996; Sitnicka et al., Blood, 87:4998-5005, 1996; Young et al., Blood, 88:1619-1631, 1996; Birkmann et al., Stem Cells, 15:18-32, 1997; Borge et al., Blood, 90:2282-2292, 1997; Katayama et al., Leuk. Lymph., 28:51-56, 1997; Ramsfjell et al., J. Immunol., 158:5169-5177, 1997; Rasko et al., Stem Cells, 15:33-42, 1997; Yoshida et al., Br. J. Haematol., 98:254-264, 1997; Kobayashi et al., Blood, 86:2494-2499, 1995; Era et al., Blood, 89:1207-1213, 1997; Alexander et al., Blood, 87: 2162-2170, 1996; Carver-Moore et al., Blood, 88: 803-808, 1996; Kobayashi et al., Blood, 88: 429436, 1996; Petzer et al., J. Exp. Med., 183: 2551-2558, 1996; Tanimukai et al., Exp. Hematol., 25: 1025-1033, 1997). For example, it has been demonstrated that recombinant human TPO (rhTPO) in combination with other cytokines is able to rescue in vitro embryonic erythropoiesis in mice lacking erythropoietin receptor (Kieran et al., Proc. Natl. Acad. Sci. USA, 93: 9126-9131, 1996). Moreover, rhTPO was shown to improve the recovery from pancytopenia in myelosuppressed mice (Kaushansky et al., Exp. Hematol., 24: 265-269, 1996; Grossmann et al., Exp. Hematol., 24: 1238-1246, 1996), and pegylated recombinant molecule related to human TPO (PEG-rhMGDF) accelerated multilineage hematopoietic recovery in both myelosuppressed mice and nonhuman primates (Akahori et al., Stem Cells, 14: 678-689, 1996; Farese et al., J. Clin. Invest., 97:2145-2151, 1996; Shibuya et al., Blood, 91: 37-45, 1998).

In summary, due to its potent ability to promote viability and suppress apoptosis, TPO alone, or in combination with other early-acting cytokines, can stimulate survival of primitive multipotent progenitor cells and trigger their division leading to expansion of the pool of long-term re-populating and culture-initiating cells. It can also induce multilineage differentiation and enhance the formation of multilineage colonies containing granulocytes, erythrocytes, macrophages, and megakaryocytes. It follows, that, in addition to its potential therapeutic role in treating thrombocytopenia, TPO can be useful for the mobilization, amplification and ex vivo expansion of stem cells and committed precursor cells for autologous and allogeneic transplantation as well as for the expansion of stem cells destined for gene therapy. Indeed, TPO has been shown to be the most potent single agent at expanding long-term culture initiating cells (LTCIC) in serum-free culture (Petzer et al., 1996, supra; Piacibello et al., Blood, 89:2644-2653, 1997).

### Thrombopoietin Receptor (MPL)

The central role of TPO in megakaryocytopoiesis and thrombopoiesis is most clearly manifested by the severe thrombocytopenic phenotype of mice rendered null for the expression of either TPO or its receptor (TPOR/MPL/c-MPL) (Gurney et al., Science, 265: 1445, 1994; Kaushansky et al., J. Clin. Invest., 96: 1683, 1995; de Sauvage et al., J. Exp. Med., 183: 651, 1996). The similarity in the phenotype of the TPO and c-MPL knockout mice shows that the system is non-redundant and that there is one receptor for TPO and one ligand for MPL. Because of this, it is presumed that binding of TPO to MPL is solely responsible for its activation.

The cell surface receptor for TPO (TPOR/MPL/c-MPL) is a product of the protooncogene c-mpl, a homologue of v-mpl, an envelope protein of the myeloproliferative leukaemia virus (MPLV) shown to induce a pan-myeloid disorder (Wendling, Virol., 149:242-246, 1986).

The human c-mpl gene codes for a protein of 635 aa having a predicted molecular weight of 71 kD (Vigon et al., Proc. Natl. Acad. Sci. USA, 89:5640-44, 1992; Mignotte et al., Genomics, 20: 5-12, 1994). Both the human and murine sources show the presence of multiple forms of MPL produced as a result of alternative mRNA splicing (Kr von dem Borne et al., Baill. Clin. Haemotol., 11: 409, 1998). Two forms, MPL-I (82-84 kD) and MPL-II (70-74 kD), are apparently expressed on the cell membrane together. Based on recent in vitro studies with murine cell lines, it appears that the long form (MPL I), a full-length product of the gene, can bind and be activated by both intact TPO and proteolytic forms of TPO. The shorter form (MPL II)that results from a 180 bp (60 aa) deletion within the extracellular domain, can bind and be activated only by proteolytic forms (Sabath et al., Blood, 88[suppl.1]: 660a, 1996). There also appears to be a soluble from (MPL-S) present in plasma and lacking transmembrane and cytoplasmic domains (Mignotte et al., Genomics, 20:5-12, 1994; Debili et al., Blood, 85:391-401, 1995).

MPL is a member of the hematopoietic growth factor receptor superfamily (Vigon et al., 1992, supra). Extracellular domains of members of this family are typically composed of multiple-sandwich modules related to the fibronectin type-III immunoglobulin fold, with a characteristic ligand-binding domain formed from two adjacent-sandwich structures (Bazan, Proc. Natl. Acad. Sci. USA, 87: 6934, 1990). The extracellular domain of MPL is predicted to have a similar structure (Vigon et al., Oncogene, 8: 2607-2615, 1993). This domain contains 465 amino acid residues and is composed of two subdomains each with four highly conserved cysteines. A comparison of murine MPL and mature human MPL, reveals that MPL is one of the most conserved members of the cytokine receptor superfamily. Indeed, the two proteins show 81% sequence identity with the most conserved region in the cytoplasmic domain showing 91% amino acid identity and with a sequence of 37 residues near the transmembrane domain being identical in both species (Vigon et al., 1993, supra).

Similarly to other hematopoietic growth factors (such as, e.g., erythropoietin [EPO], growth hormone [GH], prolactin [PRL], and granulocyte colony-stimulating factor [G-CSF]), MPL is believed to be activated by ligand-induced receptor homodimerization (Youssoufian et al., Blood, 81: 2223, 1993; Alexander et al., EMBO J., 14: 5569, 1995; Heldin, Cell, 80: 213, 1995). The earliest intracellular signals are generated by a 121 amino acid (aa) cytoplasmic domain of MPL. Using mutational analysis, this domain has been found to consist of at least two distinct functional regions involved in signal transduction and gene regulation. These regions interact with different signal transduction pathways and can be uncoupled. As is the case with many other cytokine and growth factor receptors, MPL uses the JAK-STAT signaling pathway for rapid gene regulation (Ransohoff, New Eng. J. Med., 338:616-618, 1998) as well as the Ras signal transduction cascade (Nagata and Todokoro, FEBS Lett., 377:497-501, 1995). Specifically, upon activation by ligand binding, the dimeric MPL receptor recruits two molecules of the JAK family of intracytoplasmic kinases to a distinct region of the receptor, initiating their cross-phosphorylation and activation. Once tethered to the MPL receptor and activated, the kinase also phosphorylates a subset of the intracytoplasmic tyrosine residues of the receptor, forming docking sites for a number of signaling intermediates, the latter of which are then phosphorylated (activated) by the JAKs. Besides MPL and JAK2/TYK2, the substrates phosphorylated in response to TPO binding include the nascent transcription factors STAT3 and STAT5, the adaptor proteins Grb2, Shc, and its related phosphatase SHIP, the GTP exchange factors Vav and SOS, and hematopoietic receptor-related phosphatases such as SHP-2 (Drachman et al., J. Biol. Chem., 270:4979-4982, 1995; Sattler et al., Exp. Hematol., 23:1040-1048, 1995; Hill et al., Cell Growth Diff., 7:1125-1134, 1996; Sasaki et al., Biochem. Biophys. Res. Commun., 216:338-347, 1995; Morita et al., FEBS Lett., 395:228-234, 1996; Miyakawa et al., Blood, 86:23-27, 1995; Chen et al., Blood, 86:4054-4062, 1995; Miyakawa et al., Blood, 89:2789-2798, 1997). Many of these molecules subsequently impact on distinct nuclear signaling pathways.

As many, if not all, of the proteins that support the survival, proliferation, and differentiation of hematopoietic cells use a nearly identical repertoire of signaling pathways, many investigators have proposed that the specific signal(s) generated by the binding of TPO to MPL resides in the lineage-specific distribution of receptor expression. Others believe unique signaling profiles will emerge with further study (Kaushansky, BioEssays, 21: 353-360, 1999).

### TPO Receptor Binding Sequences and Agonist Peptides

In their mutagenesis study, Kenneth et al. obtained results indicating that multiple fragments of hTPO, including sequences Asp8-Lys14 and Lys52-Lys59 are important for its receptor binding activity (J. Biol. Chem., 272: 20595-20602, 1997).

Tahara et al. (Stem Cells, 16: 54-60, 1998) have characterized a panel of antibodies raised against recombinant human TPO (rhTPO) and against synthetic peptides derived from the hTPO sequence. The epitopes for the two neutralizing antibodies (which inhibited binding of TPO to its receptor) were localized to the conserved N-terminal domain of hTPO, the amino acid sequences Asp8-Glen28 and Ala60-Arg 117, respectively, indicating that these sequences may be involved in direct binding of TPO to the MPL receptor.

Kimura et al. (J. Biochem., 122: 1046-1051, 1997) screened a random phage peptide library expressing 15 amino acid-long peptides for binding to a chimeric molecule comprising the entire extracellular domain of human MPL and the Fc region of human IgG. Peptides demonstrating the highest binding affinity were then tested for their proliferative effect on Ba/F3 cells transfected with c-mpl cDNA and for their ability to stimulate megakaryocyte differentiation of mouse bone marrow cells. Each of the active peptides contained two cysteines generally at positions 4 and 14 and bound to MPL only when the intermolecular disulfide bond between the cysteines was intact. PK1M (LQGCTLRAWRAGMC) was the most potent peptide showing an ED₅₀ of approximately 0.54 μM for stimulation of cell proliferation (compare to an ED₅₀ of approximately 0.1 nM for TPO). The ED₅₀ for stimulation of acetylcholinesterase (AchE, a marker enzyme of rodent megakaryocyte lineage cells) activity in bone marrow cells was approximately 27 μM and 0.1 nM for PK1M and TPO respectively. Kimura et al. (Biochem. Mol. Biol. Int., 44: 1203-1209, 1998) also demonstrated that the extent of PK1M peptide-induced tyrosine phosphorylation of JAK2 and activation of STAT5 in TPO-dependent Ba/F3 cells was similar to that of TPO.

See also U.S. Pat. No. 5,932,546 which discloses low molecular weight (250-5000 D) MPL agonist peptides and peptide mimetics of general sequence: XXVRD/EQXXXXX, and PCT Publication No. WO 98/25965 which describes MPL-activating dimers of cyclic peptides (MW less than 120,000 D).

By screening recombinant libraries of random peptides Cwirla et al. (Science, 276: 1696-1699, 1997) identified two families of 14-amino acid-long peptidesthat bind to human MPL receptor, compete with the TPO binding, and stimulate the proliferation of a TPO-responsive Ba/F3 cell line: (i) family 1 containing a consensus sequence VRDQIXXXL and (ii) family 2 containing a consensus sequence TLREWL (with a pair of cysteinesthat can form intramolecular disulfide-bond cyclic structures, flanking most of the peptides). The sequences of these peptides were not found in the primary sequence of TPO. Screening libraries of variants of family 2 under affinity-selective conditions yielded a peptide, AF12505 (IEGPTLRQWLAARA) that was an especially potent MPL agonist. This peptide had an IC₅₀ of 2 nM in the competitive binding assay in which TPO had an IC₅₀ of 1 nM. In a proliferation assay, the EC₅₀ values were 400 nM and 100 pM for AF12505 and TPO, respectively. Dimerization of AF12505 by a carboxyl-terminal linkage to a lysine branch produced a highly potent pseudo-symmetrical peptide dimer agonist with activity equal to TPO, with IC₅₀ and EC₅₀ values of 100 pM and 0.5 nM, respectively. This peptide dimer also stimulated the in vitro proliferation and maturation of megakaryocytes from human bone marrow cells and promoted an 80% increase in platelet count when administered to normal mice (see also U.S. Pat. No. 6,121,238 which discloses the use of AF12505 pseudosymmetrical peptide dimer and its various dipeptide derivatives in treatment of thrombocytopenia). The fact that a small peptide dimer, only one-tenth the size of TPO, can attain the affinity of receptor binding and potency of receptor activation possessed by the natural growth factor, is probably due to its ability to induce MPL dimerization.

### Immunoglobulins and Immune Response

The basic unit of antibody immunoglobulin structure is a complex of four polypeptides—two identical low molecular weight or “light” chains and two identical high molecular weight or “heavy” chains—linked together by both non-covalent associations and by disulfide bonds. Each light and heavy chain of an antibody has a variable region at its amino terminus and a constant domain at its carboxyl terminus. The variable regions are distinct for each antibody and contain the antigen binding site. Each variable domain is comprised of four relatively conserved framework regions and three regions of sequence hypervariability termed complementarity determining regions or “CDRs”. For the most part, it is the CDRs that form the antigen binding site and confer antigen specificity. The constant domains are more highly conserved than the variable regions, with slight variations due to haplotypic differences.

Based on their amino acid sequences, light chains are classified as either kappa or lambda. The constant region of heavy chains is composed of multiple domains (CH1, CH2, CH3 . . . CHx), the number depending upon the particular antibody class. The CH1 region is separated from the CH2 region by a hinge region that allows flexibility in the antibody. The variable region of each light chain aligns with the variable region of each heavy chain, and the constant region of each light chain aligns with the first constant region of each heavy chain. The CH2-CHx domains of the constant region of a heavy chain form an “Fc region” that is responsible for the effector functions of the immunoglobulin molecule, such as complement binding and binding to the Fc receptors expressed by lymphocytes, granulocytes, monocyte lineage cells, killer cells, mast cells, and other immune effector cells.

PCT Publication WO 99/25378 relates to synthebody molecules, particularly antibodies, that bind one member of a binding pair and have at least one complementarity determining region (CDR) that contains the amino acid sequence of a binding site for that member of the binding pair. The binding site is derived from the other member of the binding pair. It also relates to methods for treating, diagnosing, or screening for diseases and disorders associated with the expression of the member of the binding pair using the modified antibodies.

PCT Publication WO 99/25379 relates to vaccine compositions of antibodies in which one or more variable region cysteine residues that form intrachain disulfide bonds have been replaced with amino acid residues that do not contain a sulfhydryl group and, therefore, do not form disulfide bonds. It also relates to use of the vaccine compositions to treat or prevent certain diseases and disorders.

Deng et al. (Blood, 92: 1981-88, 1998) reported the development of a murine mAb, termed BAH-1, raised against human megakaryocytic cells that specifically recognizes the MPL receptor. This mAb showed agonist activity by stimulating megakaryocytopoiesis in vitro, and also expanded the numbers of megakaryocytic progenitor cells in myelosuppressed mice. BAH-1 antibody specifically binds to platelets and to recombinant MPL with high affinity. Similar to TPO, BAH-1 alone supported the formation of colony-forming unit-megakaryocyte (CFU-MK) colonies, and the combination of BAH-1 and IL-3 or of BAH-1 and hTPO significantly increased the number of human CFU-MK colonies. In addition, BAH-1 mAb stimulated the proliferation and maturation of primary bone marrow megakaryocytes. Thus, in the presence of BAH-1 mAb, individual large megakaryocytes as well as small megakaryocytic cells were observed in CD34⁺CD41⁺ cell cultures, and the numbers of AchE-positive megakaryocytes increased. In addition, in vivo studies showed that BAH-1, alone or in combination with TPO, expanded the numbers of megakaryocytic progenitor cells in myelo-suppressed mice. As monovalent Fab fragments of BAH-1 antibody which cannot form receptor dimers did not stimulate megakaryocytopoiesis, it is very likely that BAH-1 activates MPL in a manner similar to the naturally occurring TPO, i.e., by receptor dimerization.

Importantly, however, although BAH-1 was able to trigger cell proliferation and differentiation of human megakaryocytic precursors and immature murine megakaryocytes, by itself it failed to stimulate murine CFU-MK (colony-forming unit-granulocyte-macrophage) colony formation (i.e., its effect was seen only when administered together with IL-3 and TPO). As in a murine myelosuppressive model BAH-1 only modestly affected megakaryocytopoiesis, it is unlikely that BAH-1 can replace TPO in stimulating platelet production in vivo. Moreover, while TPO has an effect on stem cells as well as erythroid progenitors (Kaushansky et al., J. Clin. Invest., 96: 1683, 1995; Kaushansky et al., Exp. Hematol., 24: 265, 1996), BAH-1 alone had no effects on BFU-E (burst-forming unit-erythroid) and CFU-E (colony-forming unit-erythroid) colonies. The inability of BAH-1 to functionally replace TPO may have a structural basis: BAH-1 does not antagonize TPO binding to c-MPL, suggesting that they have different binding sites.

PCT Publication No. WO 99/10494 discloses the use of phage-display single chain antibodies to search for CDR candidates that would mimic TPO to activate its receptor. However, none of the agonist antibodies described in this application demonstrated levels of activity that would be similar to the levels observed with the naturally occurring or recombinant full-length TPO. Moreover, the ability of these antibodies to restore platelet levels in vivo was never tested.

Taken together, it can be concluded that prior art describes several attempts to generate TPO receptor agonist antibodies. However, the activity of these antibodies is inferior to the activity of the naturally occurring TPO or even some of the MPL agonist peptides. Accordingly, there is a current and continuing need to generate TPO receptor agonist antibodies capable of efficiently stimulating proliferation and/or differentiation and/or modulating apoptosis of hematopoietic cells (in particular, megakaryocytes or their precursors). It is critical that such antibodies, fragments or derivatives thereof, have activity which is very similar or better than the activity of the naturally occurring TPO. Such antibodies can be particularly useful for the treatment of hematopoietic disorders including thrombocytopenia caused by chemotherapy, radiation therapy or bone marrow transfusion.

The present invention addresses these and other needs in the art by providing MPL agonist constructs (e.g., synthebodies), fragments, and derivatives thereof that contain in at least one of the CDRs a sequence capable of efficient binding to TPO receptor, specifically a sequence comprising a peptide IEGPTLRQWLAARA or any variant of this peptide capable of efficient binding to the TPO receptor.

### OBJECTS OF THE INVENTION

It is therefore an object of the present invention to provide variants of an immunoglobulin variable domain. The immunoglobulin variable domain comprises (A) at least one CDR region and (B) framework regions flanking said CDR. The variant comprises (a) the CDR region having added or substituted therein at least one binding sequence and (b) the flanking framework regions, wherein the binding sequence is heterologous to the CDR and is a binding sequence from a binding site of a binding pair, and wherein said binding sequence is a TPO receptor-binding peptide.

In further embodiment, it is an object of the invention to provide a construct having (i) one or more amino acid residues in one or more of the flanking framework regions substituted or deleted, (ii) one or more amino acid residues added in one or more of the flanking framework regions, or (iii) a combination of (i) and (ii). Alternatively, the constructs have (i) one or more amino acid residues in one or more framework regions other than the framework regions flanking the CDR substituted or deleted, (ii) one or more amino acid residues added in one or more framework regions other than the framework regions flanking said CDR, or (iii) a combination of (i) and (ii). In yet another alternative, the constructs have (i) one or more amino acid residues in one or more of the flanking framework regions substituted or deleted, (ii) one or more amino acid residues added in one or more of the flanking framework regions, or (iii) a combination of (i) and (ii); and (iv) one or more amino acid residues in one or more framework regions other than the framework regions flanking the CDR substituted or deleted, (v) one or more amino acid residues added in one or more framework regions other than the framework regions flanking said CDR, or (vi) a combination of (iv) and (v).

It is also an object of the present invention to provide variants in which the CDR region has added or substituted therein at least one amino acid sequence which is heterologous to the CDR and the flanking framework regions, wherein the heterologous sequence is capable of binding to a target sequence or molecule, and wherein the heterologous sequence is a TPO receptor-binding peptide. Again, (i) one or more amino acid residues in one or more of the flanking framework regions may be substituted or deleted, (ii) one or more amino acid residues may be added in one or more of the flanking framework regions, or (iii) a combination of (i) and (ii); (i) one or more amino acid residues in one or more framework regions other than the framework regions flanking the CDR may be substituted or deleted, (ii) one or more amino acid residues may be added in one or more framework regions other than the framework regions flanking the CDR, or (iii) a combination of (i) and (ii), or (i) one or more amino acid residues in one or more of the flanking framework regions may be substituted or deleted, (ii) one or more amino acid residues may be added in one or more of the flanking framework regions, (iii) a combination of (i) and (ii); and (iv) one or more amino acid residues in one or more framework regions other than the framework regions flanking the CDR may be substituted or deleted, (v) one or more amino acid residues may be added in one or more framework regions other than the framework regions flanking the CDR, or (vi) a combination of (iv) and (v).

Further, it is an additional object of the inventionto provide molecules comprising the variants described herein. The molecules can include one or more constant domains from an immunoglobulin; a second variable domain associated with the variant such as, for example, a variable domain of a heavy chain is associated with a variable domain of a light chain in an immunoglobulin; and a second variable domain associated with the variant, with one or more constant domains from immunoglobulins.

Moreover, it is an object of the invention to provide immunoglobulins comprising a heavy chain and a light chain, wherein said heavy chain comprises a variant as described above and three constant domains from an immunoglobulin heavy chain, and the light chain comprises a second variable domain associated with the variant and a constant domain from an immunoglobulin light chain. Furthermore, the present invention provides immunoglobulins comprising a heavy chain and a light chain, wherein the light chain comprises a variant as described above and a constant domain from an immunoglobulin light chain, and the heavy chain comprises a second variable domain associated with said variant and three constant domains from an immunoglobulin heavy chain.

Isolated nucleic acids encoding these variants, molecules, and immunoglobulins are also objects of the invention, as are cells containing these nucleic acids. Recombinant non-human hosts containing these nucleic acids are also provided. Pharmaceutical compositions comprising a therapeutically or prophylactically effective amount of the variants, molecules or immunoglobulins and pharmaceutically acceptable carriers are also provided.

It is a further object to provide pharmaceutical compositions that comprise an amount of the synthetic antibody effective to bind to the TPO receptor (MPL). These compositions may further include a pharmaceutically acceptable carrier or excipient.

Additionally, it is an object of the invention to provide a synthetic antibody comprising one or more sequences IEGPTLRQWLAARA preferably in CDR 2 of a human light chain variable region. A pharmaceutical composition or vaccine composition, as set forth above, comprises this synthetic antibody.

In a further object of the invention, the invention provides a nucleic acid encoding the synthetic antibody. The invention also furnishes pharmaceutical compositions comprising the nucleic acid encoding the synthetic antibody in an amount effective to produce sufficient amounts of the antibody to bind TPO receptor (MLP). These compositions may further include a pharmaceutically acceptable carrier or excipient.

Also encompassed are expression vectors, in which the nucleic acid is operably associated with an expression control sequence. The invention extends to host cells transfected or transformed with the expression vector. The synthetic antibody or nucleic acid can be produced by isolating it from the host cells grown under conditions that permit production of the nucleic acid or expression of the synthetic antibody.

The pharmaceutical and vaccine compositions of the invention can be administered to a subject to modulate thrombopoiesis, and particularly to treat thrombocytopenia.

### SUMMARY OF THE INVENTION

Thus, the invention provides a variant of an immunoglobulin variable domain, said immunoglobulin variable domain comprising (A) at least one CDR region and (B) framework regions flanking said CDR, said variant comprising:

(a) said CDR region having added or substituted therein at least one binding sequence and

(b) said flanking framework regions, wherein said binding sequence is heterologous to said CDR and is a binding sequence from a binding site of a binding pair, and wherein said binding sequence is a thrombopoietin receptor-binding portion of thrombopoietin.

The variant described above may include (i) one or more amino acid residues in one or more of said flanking framework regions substituted or deleted, (ii) one or more amino acid residues added in one or more of said flanking framework regions, or (iii) a combination of (i) and (ii); or optionally, (i) one or more amino acid residues in one or more framework regions other than said framework regions flanking said CDR substituted or deleted, (ii) one or more amino acid residues added in one or more framework regions other than said framework regions flanking said CDR, or (iii) a combination of (i) and (ii). In addition, the variant described above may include (i) one or more amino acid residues in one or more of said flanking framework regions substituted or deleted, (ii) one or more amino acid residues added in one or more of said flanking framework regions, or (iii) a combination of (i) and (ii); and wherein (iv) one or more amino acid residues in one or more framework regions other than said framework regions flanking said CDR substituted or deleted, (v) one or more amino acid residues added in one or more framework regions other than said framework regions flanking said CDR, or (vi) a combination of (iv) and (v).

The present invention also provides a variant of an immunoglobulin variable domain, said immunoglobulin variable domain comprising (A) at least one CDR region and (B) framework regions flanking said CDR, said variant comprising:

(a) said CDR region having added or substituted therein at least one amino acid sequence which is heterologous to said CDR and

(b) said flanking framework regions,

wherein said heterologous sequence is capable of binding to a target sequence or molecule, and wherein said heterologous sequence is a thrombopoietin receptor-binding portion of thrombopoietin.

The present invention also provides a variant as described above including (i) one or more amino acid residues in one or more of said flanking framework regions substituted or deleted, (ii) one or more amino acid residues added in on or more of said flanking framework regions, or (iii) a combination of (i) and (ii); alternatively, the variant of the invention includes (i) one or more amino acid residues in one or more framework regions other than said framework regions flanking said CDR substituted or deleted, (ii) one or more amino acid residues added in one or more framework regions other than said framework regions flanking said CDR, or (iii) a combination of (i) and (ii). Still further, the invention provides a variant as described above having (i) one or more amino acid residues in one or more of said flanking framework regions substituted or deleted, (ii) one or more amino acid residues added in one or more of said flanking framework regions, (iii) a combination of (i) and (ii); and wherein (iv) one or more amino acid residues in one or more framework regions other than said framework regions flanking said CDR substituted or deleted, (v) one or more amino acid residues added in one or more framework regions other than said framework regions flanking said CDR, or (vi) a combination of (iv) and (v).

The present invention also provides a variant as described above wherein said receptor binding portion of thrombopoietin has an amino acid sequence IEGPTLRQWLAARA.

Still further, the invention provides a variant as described above wherein said receptor-binding portion of thrombopoietin is in more than one CDR.

Additionally, the invention provides a variant as described above wherein said heterologous sequence is capable of specifically binding to said target sequence or molecule.

Further, the variant of the present invention may include a CDR region selected from the group consisting of CDR 1, CDR 2 or CDR 3.

Moreover, the variant of the present invention may be an antibody.

The invention provides molecules including the variants described above, and optionally further comprising one or more of the following: (1) one or more constant domains from an immunoglobulin, and (2) a second variable domain linked to said variant. Capable of specifically binding to said target sequence or molecule. The molecule of the invention may include CDR 1, CDR2, or CDR3 in the CDR region described above. Alternatively, the molecule of the invention may be an antibody, and the antibody may be derived from a human antibody or from a chimeric or humanized antibody.

The invention also provides an immunoglobulin comprising a heavy chain and a light chain, wherein said heavy chain comprises a variant as described above and three constant domains from an immunoglobulin heavy chain, and said light chain comprises a second variable domain associated with said variant and a constant domain from an immunoglobulin light chain.

The present invention further provides an immunoglobulin comprising a heavy chain and a light chain, wherein said light chain comprises a variant as described above and a constant domain from an immunoglobulin light chain, and said heavy chain comprises a second variable domain associated with said variant and three constant domains from an immunoglobulin heavy chain.

Also provided are isolated nucleic acids encoding the variants, molecules, or immunoglobulins described above. In addition, a cell and a recombinant non-human host.

The invention further provides a pharmaceutical composition comprising a therapeutically or prophylactically effective amount of a variant, molecule, or immunoglobulin as described above, and a pharmaceutically acceptable carrier.

Still further, the invention provides a method of treating or preventing a disease in a subject in need of such treatment or prevention, said method comprising administering to said subject a disease treating or preventing effective amount of a variant, molecule, or immunoglobulin as described above, wherein (i) said disease is caused directly or indirectly by an agent, (ii) a symptom of said disease is caused by an agent, or (iii) said disease produces a physical, chemical, or biological response, wherein said agents or response include said target sequence or molecule.

### BRIEF DESCRIPTION OF THE DRAWINGS

FIGS. 1A and 1B. Consensus sequences of (A) the heavy chain variable region and (B) the light variable region.

FIG. 2. Diagram of PCR knitting strategy.

FIG. 3. Graphical representation of the results of a cell binding experiment of synthebody binding activity. Briefly, synthebody binding to c-MPL (TPO receptor) was examined by FACS analysis of leukemia cell lines TF-1 (previously shown to express c-MPL) and KG-1 cells (reported to have low/undetectable levels of c-MPL). Varying concentrations of human TPO synthebodies were incubated with leukemia cells in vitro, washed and antibody binding was detected using a FITC labeled anti-human IgG secondary antibody. Synthebody binding was examined for TPO VLCDR2 (-▪-▪-▪-), TPO VHCDR3 (-▴-▴-▴-), TPO VLCDR1 (- [symbol] - [symbol] - [symbol] -), TPO VHCDR1 (-□-□-□-), and Human consensus (-●-●-●-) and the results of these binding studies to TF-1 and KG-1 cells are depicted in FIG. 3.

### DETAILED DESCRIPTION OF THE INVENTION

In one aspect, the present invention provides a construct that contains a TPO receptor binding sequence in a CDR (flanked by framework regions of a variable region), wherein binding of the construct precludes binding of TPO to the same receptor. According to a preferred aspect of the present invention, the TPO receptor binding region of the construct comprises a-monomer or dimer of the sequence IEGPTLRQWLAARA or any variant of this sequence capable of efficient binding to the TPO receptor, such variant being produced by any combination of amino acid substitutions, deletions or insertions. The invention further provides pharmaceutical compositions that comprise an amount of the construct effective to activate TPO receptor (MPL) in vivo, leading to stimulation of proliferation and/or differentiation and/or modulation of apoptosis of hematopoietic cells, especially platelet progenitor cells, and a pharmaceutically acceptable carrier or excipient.

Recombinant nucleic acids, particularly DNA molecules, provide for efficient expression of the foregoing constructs. In one aspect, this invention provides a nucleic acid encoding the construct. Also encompassed are expression vectors in which the nucleic acid is operably associated with an expression control sequence. The invention extends to host cells transfected or transformed with the expression vector. The construct can be produced by isolating it from the host cells grown under conditions that permit expression of the construct.

The invention also furnishes a pharmaceutical composition comprising the expression vector that expresses the construct in an amount effective to express sufficient construct to activate TPO receptor (MPL) in vivo, leading to stimulation of proliferation and/or differentiation and/or modulation of apoptosis of hematopoietic cells, especially platelet progenitor cells, and a pharmaceutically acceptable carrier or excipient.

The pharmaceutical compositions of the invention can be administered to a subject to treat hematopoietic or immune disorders, and particularly thrombocytopenia-associated bone marrow hypoplasia following chemotherapy, radiation therapy or bone marrow transfusion; disseminated intravascular coagulation (DIC); immune thrombocytopenia (including HIV-induced ITP and non-HIV-induced ITP); chronic idiopathic thrombocytopenia; congenital thrombocytopenia; myelodysplasia, and thrombotic thrombocytopenia. In addition, compositions comprising synthebodies of the invention can be used for the mobilization, amplification and ex vivo expansion of stem cells and committed precursor cells for autologous and allogeneic transplantation as well as for the expansion of stem cells destined for gene therapy. The synthebodies of the invention are also useful as diagnostic or analytical reagents for studying the function of TPO and its receptor in vitro and in vivo.

An immunoglobulin construct, e.g., an antibody having agonist activity that stimulates MPL can serve as a therapeutic option to the use of naturally occurring or recombinant fill-length TPO or MPL agonist peptides in situations in which a prolonged half-life is needed and in which less frequent administration is desired. In addition, MPL agonist synthebodies should prove useful for improving understanding of the biology of megakaryocytopoiesis. For example, the use of such synthebodies may significantly facilitate studies aimed at understanding the structure-function aspects of the MPL receptor (e.g., a more detailed mapping of the binding site of these synthebodies on MPL may help define the sequences and confirmation of the native receptorthat are necessary and sufficient for activation and subsequent signal transduction).

The present invention is based, in part, on the development of an MPL-binding synthetic antibody by insertion of a receptor-binding sequence (i.e., IEGPTLRQWLAARA or its derivative) into a CDR of a consensus variable region (see Cwirla et al., Science, 276:1696-1699, 1997; U.S. Pat. No. 6,121,238; PCT Publication WO 99/25378). This synthebody binds to MPL with high affinity leading to activation of MPL-mediated signal transduction pathway(s) that, in turn, stimulates proliferation and/or differentiation and/or modulates apoptosis of hematopoietic cells, especially platelet progenitor cells. Optimally, it mediates suppression of thrombocytopenia in vivo.

The term “construct” refers to the variant of a variable domain of an immunoglobulin superfamily protein, including molecules comprising such variants, described herein. The immunoglobulin superfamily is well known, and includes antibody/B-cell receptor proteins, T lymphocyte receptor proteins, and other proteins mentioned infra (see, Paul, Fundamental Immunology, 3^{rd} Ed.). The modification refers to insertion into or substitution of a portion of the immunoglobulin superfamily protein sequence with a heterologous amino acid sequence or heterologous binding sequence. The site of substitution in the immunoglobulin superfamily protein corresponds to a binding-accessible portion of the region of the immunoglobulin superfamily protein, e.g., a region that corresponds to an antibody variable region, and more particularly a portion corresponding to a CDR of an antibody variable region.

A “synthebody” (for synthetic antibody) is a specific example of a construct of the invention that includes an antibody variable region. It may also include regions corresponding to an antibody constant region or regions, or be associated with one or more other immunoglobulin family polypeptides, such as an antibody Fv heterodimer, an antibody tetramer, a T lymphocyte receptor heterodimer, etc. Embodiments described below are illustrative of the variants and molecules of the present invention in that the variants are included in synthebodies and synthebodies are a type of molecule that includes the variants. The term “synthebody” thus refers to an illustrative example of a type of construct of the invention.

The term “heterologous” refers to a combination of elements not naturally occurring in a particular locus. For example, heterologous DNA refers to DNA not naturally located in the cell or in a particular chromosomal site of the cell. A heterologous expression regulatory element is such an element operatively associated with a different gene than the one it is operatively associated with in nature. In the context of the present invention, a construct coding sequence is heterologous to the vector DNA in which it is inserted for cloning or expression and it is heterologous to a host cell containing such a vector in which it is expressed, e.g., a CHO cell. Moreover, the constructs of the present invention contain a heterologous DNA, amino acid, or binding sequence.

The “heterologous amino acid (or binding) sequence” (also “binding sequence”) refers to the desired binding segment of a polypeptide, e.g., the portion of a polypeptide (protein or peptide) that binds to a receptor. As used in this application, the term refers to the sequence of a peptide that binds to the TPO receptor.

A “target receptor” or “target binding partner” (also simply “target”) is a molecule that is recognized and specifically bound by a construct. In particular, the target receptor is MPL/TPO receptor.

The terms “agonist” and “agonistic” when used herein refer to a molecule that is capable of, directly or indirectly, substantially inducing, promoting or enhancing cytokine biological activity or cytokine receptor activation. Accordingly, “agonist antibodies” (aAb) are antibodies or fragments thereof that possess the property of binding to a cytokine superfamily receptor and causing the receptor to transduce a differentiation and/or proliferation and/or survival signal. Included within the definition of transducing a survival signal is a signal that modulates cell survival or death by apoptosis. As disclosed herein, the agonist antibodies of this invention are capable of stimulating or modulating proliferation and/or differentiation and/or survival at a concentration equal to or not less that of the natural in vivo ligand (TPO).

“Activate a receptor”, as used herein, is used interchangeably with transduce a growth and/or proliferation and/or differentiation and/or survival signal.

“Cytokine” is a generic term for a group of proteins released by one cell population that act on another cell population as intercellular mediators. Examples of such cytokines are lymphokines, monokines, and traditional polypeptide hormones. Included among the cytokines are growth hormones (GH), insulin-like growth factors (IGF), parathyroid hormone, thyroxine, insulin, insulin, relaxin, follicle stimulating hormone (FSH), thyroid stimulating hormone (TSH), leutinizing hormone (LH), hematopoietic growth factor, hepatic growth factor, fibroblast growth factors (FGF), prolactin, placental lactogen, tumor necrosis factors (TNF), mullerian-inhibiting substance, mouse gonadotropin-associated peptide, inhibin, activin, vascular endothelial growth factor, integrin, nerve growth factors (NGF), platelet growth factor, transforming growth factors (TGF), erythropoietin (EPO), osteoinductive factors, interferons (IFN), colony stimulating factors (CSF), thrombopoietin (TPO), interleukins (IL), leukemia inhibitory factor (LIF), kit-ligand, etc. As used herein the foregoing terms are meant to include both naturally occurring and recombinant proteins. Similarly, the terms are intended to include biologically active equivalents, e.g., differing in amino acid sequence by one or more amino acids or in type or extent of glycosylation.

“Cytokine superfamily receptors” and “hematopoietic growth factor superfamily receptors” are used interchangeably herein and are a group of closely related glycoprotein cell surface receptors that share considerable homology (including frequently a WSXWS domain) and are generally classified as members of the cytokine receptor superfamily (see, e.g., Nicola et al., Cell, 67: 1-4, 1991; Skoda et al., EMBO J., 12: 2645-2653, 1993). Generally, these receptors have as their natural ligands interleukins (IL) or colony-stimulating factors (CSF). Members of the superfamily include, but are not limited to, receptors for: IL-2 (Hatakeyama et al., Science, 244: 551-556, 1989; Takeshita et al., Science, 257: 379-382, 1991), IL-3 (Itoh et al., Science, 247: 324-328 1990; Gorman et al., Proc. Natl. Acad Sci. USA, 87: 5459-5463, 1990; Kitarnura et al., Cell, 66: 1165-1174, 1991; Kitamura et al., Proc. Natl. Acad. Sci. USA, 88: 5082-5086, 1991), IL-4 (Mosley et al., Cell, 59: 335-348, 1989), IL-5 (Takaki et al., EMBO J., 9: 4367-4374, 1990; Tavernier et al., Cell, 66: 1175-1184, 1991), IL-6 (Yamasaki et al., Science, 241: 825-828, 1988; Hibi et al., Cell, 63: 1149-1157, 1990), IL-7 (Goodwin et al., Cell, 60: 941-951, 1990), IL-9 (Renault et al., Proc. Natl. Acad. Sci. USA, 89: 5690-5694, 1992), granulocyte-macrophage colony-stimulating factor (GM-CSF) (Gearing et al, EMBO J., 8: 3667-3676, 1991; Hayashida et al., Proc. Natl. Acad. Sci. USA, 244: 9655-9659, 1990), granulocyte colony-stimulating factor (G-CSF) (Fukunaga et al, Cell, 61: 341-350, 1990; Fukunaga et al, Proc. Natl. Acad. Sci. USA, 87: 8702-8706, 1990, Larsen et al., J Exp. Med., 172: 1559-1570, 1990), erythropoietin (EPO) (D'Andrea et al., Cell, 57: 277-285, 1989; Jones et al., Blood, 76: 31-35, 1990), leukemia inhibitory factor (LIF) (Gearing et al., EMBO J., 10: 2839-2848, 1991), oncostatin M (OSM) (Rose et al., Proc. Natl. Acad. Sci. USA, 88: 8641-8645, 1991), prolactin (Boutin et al., Proc. Natl. Acad. Sci. USA, 88: 7744-7748, 1988; Edery et al., Proc. Natl. Acad. Sci. USA, 86: 2112-2116, 1989), growth hormone (GH) (Leung et al., Nature, 330: 537-543, 1987), ciliary neurotrophic factor (CNTF) (Davis et al., Science, 253:59-63, 1991) and thrombopoietin (TPO) (Souyri et al, Cell, 63: 1137, 1990; Vigon et al., Proc. Natl. Acad. Sci., 89: 5640, 1992).

“Thrombocytopenia” in humans is defined as a platelet count below 150×10⁹ per liter of blood.

“Thrombopoietic activity” is defined as biological activity that consists of stimulating proliferation and/or differentiation and/or modulating apoptosis of megakaryocytes or megakaryocyte precursors into the platelet producing form of these cells. This activity may be measured in various assays including without limitation (i) in vivo platelet rebound synthesis assay (³⁵S incorporation), (ii) immunodetection of the induction of platelet-specific cell surface antigens (e.g., GPIIbIIIa), and (iii) detection (using, e.g., radioactive or fluorescent label incorporation) of induction of chromosomal polyploidization in megakaryocytes.

The terms “thrombopoietin receptor” or “TPO receptor ” or “TPOR” are used interchangeably to refer to a mammalian polypeptide receptor that, when activated by a ligand binding thereto, causes “thrombopoietic activity” in a cell or mammal, including a human. Besides naturally occurring receptors (including alleles and isoforms), the terms “thrombopoietin receptor” or “TPO receptor ” or “TPOR” encompass various derivatives, such as fragments, analogues, epitope tagged versions, chimeric versions and mixtures of these forms. A preferred TPO receptor of the present invention is c-MPL, a member of the cytokine receptor superfamily. Accordingly, the terms “c-MPL” or “MPL” are used interchangeably with the terms “thrombopoietin receptor” or “TPO receptor ” or “TPOR”.

The terms “MPL ligand ” or “MPL ligand polypeptide ” or “TPO receptor ligand” are used interchangeably herein and include any peptide (e.g., IEGPTLRQWLAARA) or protein (e.g, synthebody) that possesses the property of binding to MPL receptor. Thrombopoietin (TPO) is a naturally occurring MPL ligand. This definition encompasses peptides and proteins isolated from natural sources or prepared by recombinant or synthetic methods. The terms “MPL ligand” or “MPL ligand polypeptide ” or “TPO receptor ligand” include variant forms, such as fragments, alleles, isoforms, analogues, chimera thereof and mixtures of these forms. Preferably, the MPL ligand is a compound having thrombopoietic activity (i.e., capable of increasing serum platelet counts in a mammal by at least 10%, or more preferably by 50%, and most preferably capable of elevating platelet counts in a human to greater than about 150×10⁹ per liter of blood).

“MPL ligand analogues” include covalent modification of MPL ligand produced by linking it to one of a variety of non-proteinaceous polymers, e.g., polyethylene glycol, polypropylene glycol, or poyoxyalkylenes, in the manner set forth in U.S. Pat. Nos. 4,640,835; 4,496,689; 4,301,144; 4,670,417; 4,791,192 and 4,179,337. TPO polypeptides covalently linked to the forgoing polymers are referred to herein as pegylated TPO.

Still other preferred MPL ligands of this invention include MPL ligand sequence variants and chimeras. As disclosed herein, preferred MPL ligand sequence variants and chimeras provide an improved hematopoietic (e.g., thrombopoietic) activity and serum half-life and possess an amino acid sequence having at least 90% amino acid sequence identity with the original MPL ligand and most preferably at least 95%. A preferred chimera is a fusion between MPL ligand or a fragment thereof with a heterologous polypeptide.

The term “CDR” refers to a part of the variable region of an immunoglobulin family protein that confers binding specificity, e.g., antibody specificity for antigen. In antibodies, CDRs are highly variable and accessible. The site of introduction of the thrombopoietin receptor binding sequence is termed herein a “CDR”.

The term “framework region” refers to the part of the modified immunoglobulin molecule corresponding to an antibody framework region, as defined in the art. Sequences flanking the CDR are termed herein “framework regions of a variable region”.

The term “flanked” and “flanking” refers to the amino acids that are connected to or are connected by spacing amino acids to the protein sequence of the CDR. “Spacing amino acids” (or a “spacer group”) are amino acids that are not found in the native framework sequence or the CDR or the substituted sequence, nor do they independently confer any binding activity on the modified variable region. They may be included to preserve or ensure a proper variable region conformation and orientation of the CDR or substituted heterologous amino acid sequence.

As disclosed herein, the constructs of this invention are substantially homogeneous immoglobulin family proteins, that possess the property of efficiently binding to the TPO receptor (TPOR/MPL/c-MPL) and transducing a proliferation and/or growth and/or differentiation and/or survival signal. Such signal transduction activity of the synthebodies of the present invention may be determined, e.g., by (i) detecting increased polyploidization by stimulation of incorporation of labeled nucleotides (³H-thymidine) into the DNA of cells transfected with human MPL (e.g., Ba/F3); (ii) measuring induction of the platelet-specific antigen (e.g., GPIIbIIIa) expression; (iii) detecting (e.g., using KIRA ELISA) changes in the level of phosphorylation of the MPL-derived chimeric receptor (e.g., c-MPL-Rse.gD); (iv) analyzing proliferation of c-MPL/Mab HU-03 cells; and (v) performing a liquid suspension megakaryocytopoiesis assay.

Preferred MPL agonist antibodies of this invention are also capable of stimulating proliferation and/or growth and/or survival of various hematopoietic progenitor cells (e.g., megakaryocytes, CD34+ cells, granulocytic macrophage progenitors, and erythroid progenitors) or platelet-producing differentiation of megakaryocytes at a concentration equal to or not less than that of naturally occurring TPO. The synthebodies of this invention possess hematopoietic, especially megakaryocytopoietic or thrombocytopoietic activity—namely, they are capable of stimulating proliferation, growth and/or differentiation and/or modulate apoptosis of immature megakaryocytes or their predecessors into the mature platelet-producing form that demonstrate a biological activity equal to that of rhTPO. Most preferred antibodies of this invention are human antibodies including full length antibodies having an intact human Fc region and including fragments thereof having hematopoietic, megakaryocytopoietic and/or thrombopoietic activity. Exemplary fragments having the above described biological activity include; Fv, scFv, F(ab′), F(ab′)2. These scFvs can be affinity matured by mutating amino acid residues in one or more of the CDRs or in the framework regions between the CDRs.

The phrase “pharmaceutically acceptable”, whether used in connection with the pharmaceutical compositions of the invention or vaccine compositions of the invention, refers to molecular entities and compositions that are physiologically tolerable and do not typically produce untoward reactions when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin, 18^{th} Edition.

The term “about” or “approximately” will be known to those skilled in the art in light of this disclosure. Preferably, the term means within 20%, more preferably within 10%, and more preferably still within 5% of a given value or range. Alternatively, especially in biological systems, the term “about” preferably means within about a log (i.e., an order of magnitude) preferably within a factor of two of a given value, depending on how quantitative the measurement.

### Molecular Biology—Definitions

A “coding sequence” or a sequence “encoding” an expression product, such as a RNA, polypeptide, protein, or enzyme, is a nucleotide sequence that, when expressed, results in the production of that RNA, polypeptide, protein, or enzyme, i e., the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins, and may or may not include regulatory DNA sequences, such as promoter sequences, that determine for example the conditions under which the gene is expressed. The transcribed region of a gene can include 5′- and 3′-untranslated regions (UTRs) and introns in addition to the translated (coding) region.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of or “operably associated with” transcriptional and translational control sequences in a cell. RNA polymerase transcribes the coding sequence into mRNAthat is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a mRNA or a protein. The expression product itself, e.g. the resulting mRNA or protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell. “Conditions that permit expression” in vitro are culture conditions of temperature (generally about 37^{?} C.), humidity (humid atmosphere), carbon dioxide concentration to maintain pH (generally about 5% CO₂ to about 15% CO₂), pH (generally about 7.0 to 8.0, preferably 7.5), and culture fluid components that depend on host cell type. In vivo, the conditions that permit expression are primarily the health of the non-human transgenic animalthat depends on adequate nutrition, water, habitation, and environmental conditions (light-dark cycle, temperature, humidity, noise level). In either system, expression may depend on a repressor or inducer control system, as well known in the art.

The term “transfection” means the introduction of a “foreign” (i.e., extrinsic or extracellular) gene, DNA or RNA sequence into a host cell, so that the host cell will express the introduced gene or sequence to produce a desired substance, typically a protein or enzyme encoded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transfected” and is a “transfectant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transfect the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA segment that can be inserted into a vector or into another piece of DNA at a defined restriction site. Preferably, a cassette is an “expression cassette” in which the DNA is a coding sequence or segment of DNA that codes for an expression product that can be inserted into a vector at defined restriction sites. The cassette restriction sites generally are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid” that generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. A large number of vectors, including plasmid and fungal vectors, have been described for replication and/or expression in a variety of eukaryotic and prokaryotic hosts. Non-limiting examples include pKK plasmids (Amersham Pharmacia Biotech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra. The host cell may be found in vitro, i.e., in tissue culture, or in vivo, i.e., in a microbe, plant or animal.

The term “expression system” means a host cell and compatible vector under suitable conditions, e.g., for the expression of a protein coded for by foreign DNA carried by the vector and introduced to the host cell. Common expression systems include E. coli host cells and plasmid vectors, insect host cells and Baculovirus vectors, and mammalian host cells and vectors. In a specific embodiment, the synthebody is expressed in COS-1 or CHO cells. Other suitable cells include NSO cells, HeLa cells, 293T (human kidney cells), mouse primary myoblasts and NIH 3T3 cells.

The terms “mutant” and “mutation” mean any detectable change in genetic material, e.g., DNA, or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. The term “variant” may also be used to indicate a modified or altered gene, DNA sequence, enzyme, cell, etc., i.e., any kind of mutant.

“Sequence-conservative variants” of a polynucleotide sequence are those in which a change of one or more nucleotides in a given codon position results in no alteration in the amino acid encoded at that position.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, more preferably at least 85%, and even more preferably at least 90%, and that has the same or similar properties or functions as the native or parent protein or enzyme to which it is compared.

As used herein, the term “oligonucleotide” refers to a nucleic acid, generally of at least 10, preferably at least 15, and more preferably at least 20 nucleotides, preferably no more than about 100 nucleotides, that is hybridizable to a genomic DNA molecule, a cDNA molecule, or an mRNA molecule having a sequence of interest. Oligonucleotides can be labeled, e.g., with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of the synthebody, or to detect the presence of nucleic acids encoding the synthebody. In a further embodiment, an oligonucleotide of the invention can form a triple helix with a synthebody-encoding DNA molecule, e.g., for purification purposes. Generally, oligonucleotides are prepared synthetically, preferably on a nucleic acid synthesizer. Accordingly, oligonucleotides can be prepared with non-naturally occurring phosphoester analog bonds, such as thioester bonds, etc.

### Constructs

The constructs of the invention can be derived from any type of immunoglobulin molecule, for example, but not limited to, antibodies, T lymphocyte receptors, cell-surface adhesion molecules such as the co-receptors CD4, CD8, CD19, and the invariant domains of MHC molecules. In a preferred embodiment of the invention, the construct is derived from an antibodythat can be any class of antibody, e.g., an IgG, IgE, IgM, IgD or IgA, preferably, the antibody is an IgG. Such antibodies may be in membrane bound (B cell receptor) or secreted form, preferably secreted. Additionally, the antibody may be of any subclass of the particular class of antibodies. In another specific embodiment, the construct is derived from a T lymphocyte receptor.

CDR-grafted variable region genes have been constructed by various methods such as site-directed mutagenesis as described in Jones et al., Nature, 1986, 321:522; Riechmann et al., Nature, 1988, 332:323; in vitro assembly of entire CDR-grafted variable regions (Queen et al., Proc. Natl. Acad. Sci. USA, 1989, 86:10029); and the use of PCR to synthesize CDR-grafted genes (Daugherty et al., Nucleic Acids Res., 1991, 19:2471). CDR-grafted antibodies are generated in which the CDRs of the murine monoclonal antibody are grafted onto the framework regions of a human antibody. Following grafting, most antibodies benefit from additional amino acid changes in the framework region to maintain affinity, presumably because framework residues are necessary to maintain CDR conformation, and some framework residues have been demonstrated to be part of the antigen combining site. Such CDR-grafted antibodies have been successfully constructed against various antigens, for example, antibodies against IL-2 receptor as described in Queen et al. (Proc. Natl. Acad. Sci. USA, 1989, 86:10029), antibodies against cell surface receptors-CAMPATH as described in Riechmann et al. (Nature, 1988, 332:323); antibodies against hepatitis B in Co et al. (Proc. Natl. Acad. Sci. USA, 1991, 88:2869); as well as against viral antigens of the respiratory syncitial virus in Tempest et al. (BioTechnology, 1991, 9:267). Thus, in specific embodiments of the invention, the construct comprises a variable domain in which at least one of the framework regions has one or more amino acid residues that differ from the residue at that position in the naturally occurring framework region. The techniques employed in creating CDR-grafted antibodies can be adapted for use in constructs of the invention.

The heterologous amino acid sequence can be inserted into any one or more of the CDR regions of the variable domain variant. It is within the skill in the art to insert the binding site into different CDRs of the variable domain and then screen the resulting modified constructs for the ability to bind to the binding partner of the heterologous amino acid sequence. Thus, one can determine which CDR optimally contains the binding site. In specific embodiments in which the construct is an antibody, a CDR of either the heavy or light chain variable region is modified to contain the amino acid sequence of the binding site. In another specific embodiment, the construct contains a variable domain in which the first, second, or third CDR of the heavy chain variable region or the first, second, or third CDR of the light chain variable region contains the amino acid sequence of the binding site. In another embodiment of the invention, more than one CDR contains the amino acid sequence of the binding site or more than one CDR each contains a different binding site sequence for the same molecule or contains a different binding site sequence for a different molecule. In particular embodiments, two, three, four, five or six CDRs (per heavy chain—light chain pair) are engineered to contain a peptide shown to bind to the TPO receptor. Corresponding modifications are also contemplated for other immunoglobulin superfamily protein-derived constructs of the invention.

In specific embodiments of the invention, the binding site amino acid sequence is either inserted into the CDR without replacing any of the amino acid sequence of the CDR itself or, alternatively, the binding site amino acid sequence replaces all or a portion of the amino acid sequence of the CDR.

Relative efficacy of an TPO receptor-binding construct can be evaluated by direct binding assays, such as ELISA, Western blotting, direct binding to cells (that can be detected by radiolabelling or fluorescence labeling) and the like; inhibition assays, e.g., with labeled soluble TPO; and functional assays, including profiferation and differentation of megakaryocytes, and in vivo models of thrombocytopension.

After preparing constructs containing modified variable domains, the constructs, can be further altered and screened to select an antibody having higher affinity or specificity. Constructs having higher affinity or specificity for the target binding partner may be generated and selected by any method known in the art. For example, but not by way of limitation, the nucleic acid encoding the construct can be mutagenized, either randomly, i.e., by chemical or site-directed mutagenesis, or by making particular mutations at specific positions in the nucleic acid encoding the construct, and then screening the construct expressed from the mutated nucleic acid molecules for binding affinity for the target molecule. Screening can be accomplished by testing the expressed antibody constructs individually or by screening a library of the mutated sequences, e.g., by phage display techniques (see, e.g., U.S. Pat. Nos. 5,223,409; 5,403,484; and 5,571,698; PCT Publication WO 92/01047) or any other phage display technique known in the art.

Accordingly, in a specific embodiment, the construct may have a higher specificity or affinity for its target binding partner than a naturally occurring antibody that specifically binds the same antigen. In another embodiment, the modified antibody exhibits a binding constant for target binding partner ranging from about 1×10⁶ to about 1×10¹⁴ M⁻¹.

The constructs of the invention may also be further modified in any way known in the art, e.g., for the modification of antibodies as long as the further modification does not completely prevent binding of the construct to the particular binding partner. In particular, the constructs of the invention may have one or more amino acid substitutions, deletions, or insertions besides the insertion into or replacement of CDR sequences with the binding sequence. Such amino acid substitutions, deletions, or insertions can be any substitution, deletion, or insertion that does not prevent the specific binding of the construct to the target binding partner. For example, such amino acid substitutions include substitutions of functionally equivalent amino acid residues. One or more amino acid residues can be substituted by another amino acid of a similar polarity that acts as a functional equivalent resulting in a silent alteration. Substitutes for an amino acid may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

Additionally, one or more amino acid residues can be substituted by a nonclassical amino acid or chemical amino acid analogs, introduced as a substitution or addition into the immunoglobulin sequence. Non-classical amino acids include but are not limited to the D-isomers of the common amino acids, alpha-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, beta-alanine, fluoro-amino acids, designer amino acids such as beta-methyl amino acids, C-alpha-methyl amino acids, N-alpha-methyl amino acids, and amino acid analogs in general. Furthermore, the amino acid can be D (dextrorotary) or L (levorotary).

### Preferred Immunoglobulin Family Proteins

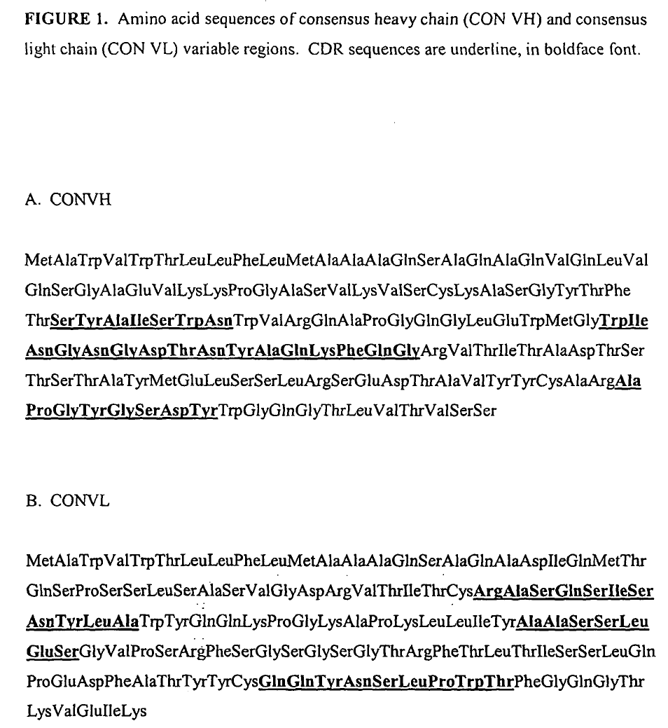

The immunoglobulin molecule modified to generate the constructs is preferably a monoclonal antibody. The antibody that is modified may be a naturally occurring or previously existing antibody, or may be synthesized from known antibody consensus sequences, such as the consensus sequences for the light and heavy chain variable regions in FIGS. 1A and 1B, or any other antibody consensus or germline (i.e., unrecombined genomic sequences) sequences (e.g., those antibody consensus and germline sequences described in Kabat et al., 1991, Sequences of Proteins of Immunological Interest, 5^{th} edition, NIH Publication No. 91-3242, pp. 2147-2172).

The invention further provides constructs that are modified chimeric or humanized antibodies. A chimeric antibody is a molecule in which different portions of the antibody molecule are derived from different animal species, such as those having a variable region derived from a murine mAb and a constant region derived from a human immunoglobulin constant region. Techniques have been developed for the production of chimeric antibodies (Morrison et al., Proc. Natl. Acad. Sci. USA, 1984, 81:6851-6855; Neuberger et al., Nature, 1984, 312:604-608; Takeda et al., Nature, 1985, 314:452454; International Patent Application No. PCT/GB85/00392) by splicing the genes from a mouse antibody molecule of appropriate antigen specificity together with genes from a human antibody molecule of appropriate biological activity. In a specific embodiment, the synthebody is a chimeric antibody containing the variable domain of a non-human antibody and the constant domain of a human antibody.

In another embodiment, the construct is derived from a humanized antibody, in which the CDRs of the antibody (except for the one or more CDRs containing the heterologous binding sequence) are derived from an antibody of a non-human animal and the framework regions and constant region are from a human antibody (see, U.S. Pat. No. 5,225,539).

As noted above, the construct can be derived from a human monoclonal antibody. The creation of completely human monoclonal antibodies is possible through the use of transgenic mice. Transgenic mice in which the mouse immunoglobulin gene loci have been replaced with human immunoglobulin loci provide in vivo affinity-maturation machinery for the production of human immunoglobulins.

The term “native antibodies and immunoglobulins” refers to usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J Mol. Biol., 186: 651-663, 1985; Novotny and Haber, Proc. Natl. Acad. Sci. USA, 82: 45924596, 1985).

The term “variable” refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRS) or hypervariable regions both in the light chain and the heavy chain variable domains. The more highly conserved portions of variable domains are called the framework (FR). The variable domains of native heavy and light chains each comprise four FR regions, largely adopting a—sheet configuration, connected by three CDRsthat form loops connecting, and in some cases forming part of, the -sheet structure. The CDRs in each chain are held together in close proximity by the FR regions and, with the CDRs from the other chain, contribute to the formation of the antigen binding site of antibodies (see Kabat et al., Sequences of Proteins of Immunological Interest, National Institute of Health, Bethesda, Md., 1987). The constant domains are not involved directly in binding an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa and lambda, based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG and IgM, and several of these may be further divided into subclasses (isotypes), e.g., IgG-1, I-G-2, IgG-3, and IgG-4; IgA-1 and IgA-2. The heavy chain constant domains that correspond to the different classes of immunoglobulins are called alpha, delta, epsilon, gamma, and mu, respectively. The subunit structures and three-dimensional configurations of different classes of immunoglobulins are well known.

Papain digestion of antibodies produces two identical antigen binding fragments, called “Fab” fragments, each with a single antigen binding site, and a residual “Fc” fragment that contains regions involved in the effector functions of the immunoglobulin molecule, such as complement binding and binding to the Fc receptors expressed by lymphocytes, granulocytes, monocyte lineage cells, killer cells, mast cells, and other immune effector cells. Pepsin treatment of antibodies yields a F(ab′)2 fragment that has two antigen combining sites and is still capable of cross-linking antigen. The Fab fragment also contains the constant domain of the light chain and the first constant domain (CH1) of the heavy chain. Fab″ fragments differ from Fab fragments by the addition of a few residues at the C-terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other, chemical couplings of antibody fragments are also known.

“Fv” is the minimum antibody fragment that contains a complete antigen recognition and binding site. This region consists of a dimer of one heavy and one light chain variable domain in tight, non-covalent association. It is in this configuration that the three CDRs of each variable domain interact to define an antigen binding site on the surface of the VH-VL dimer. Collectively, the six CDRs confer antigen binding specificity to the antibody. However, even a single variable domain (or half of an Fv comprising only three CDRs specific for an antigen) has the ability to recognize and bind antigen, although at a lower affinity than the entire binding site.

The term “antibody” or “Ab” is used in the broadest sense and specifically covers single monoclonal antibodies (including agonist and antagonist antibodies), antibody compositions with polyepitopic specificity, as well as antibody fragments (e.g., Fab, F(ab′)2, scFv and Fv), so long as they exhibit the desired biological activity.

The term “monoclonal antibody” or “mAb” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal, pAb) antibody preparations that typically include different antibodies directed against different determinants (epitopes), each monoclonal antibody is directed against a single determinant on the antigen. In addition to their specificity, the monoclonal antibodies are advantageous in that they are synthesized by the hybridoma, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein (Nature, 256: 495, 1975), or may be made by recombinant DNA methods (see, e.g., U.S. Pat. No. 4,816,567).

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity, e.g., binding to and activating MPL (U.S. Pat. No. 4,816,567; Morrison et al., Proc. Natl. Acad. Sci. USA, 81: 6851-6855, 1984).

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Furthermore, a humanized antibody may comprise residues that are found neither in the recipient antibody nor in the imported CDR or framework sequences. These modifications are made to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin. For further details see Jones et al, Nature, 321: 522-525, 1986; Reichmann et al., Nature, 332: 323-329, 1988; Presta, Curr. Opin. Struct. Biol., 2: 593-596, 1992.

“Single-chain Fv” or “sFv” antibody fragments comprise the VH and VL domains of an antibody, wherein these domains are present in a single polypeptide chain. Generally, the Fv polypeptide further comprises a polypeptide linker between the VH and VL domains that enables the sFv to form the desired structure for antigen binding (for a review see Pluckthun, In: The Pharmacology of Monoclonal Antihodies, vol. 113, Rosenburg and Moore eds., Springer-Verlag, New York, pp. 269-315, 1994).

The term “diabodies” refers to small antibody fragments with two antigen-binding sites, which fragments comprise a heavy chain variable domain (VH) connected to a light chain variable domain (VL) in the same polypeptide chain. By using a linker that is too short to allow pairing between the two domains on the same chain, the domains are forced to pair with the complementary domains of another chain and create two antigen-binding sites. Diabodies are described more fully in, e.g., European Patent No. EP 404,097; PCT Publication No. WO 93/11161; Hollinger et al., Proc. Natl. Acad. Sci. USA, 90: 6444-6448, 1993.

The expression “linear antibodies” when used throughout this application refers to the antibodies described by Zapata et al. (Protein Eng., 8: 1057-1062, 1995). Briefly, these antibodies comprise a pair of tandem VH-CH1-VH-CH1 segments that form a pair of antigen binding regions. Linear antibodies can be bispecific or monospecific.

A “variant” antibody, refers herein to a molecule that differs in amino acid sequence from a “parent” antibody amino acid sequence by virtue of addition, deletion and/or substitution of one or more amino acid residue(s) in the parent antibody sequence. In the preferred embodiment, the variant comprises one or more amino acid substitution(s) in one or more hypervariable region(s) of the parent antibody. For example, the variant may comprise at least one, e.g., from about one to about ten, and preferably from about two to about five, substitutions in one or more hypervariable regions of the parent antibody. Ordinarily, the variant will have an amino acid sequence having at least 75% amino acid sequence identity with the parent antibody heavy or light chain variable domain sequences, more preferably at least 80%, more preferably at least 85%, more preferably at least 90%, and most preferably at least 95%. Identity or homology with respect to this sequence is defined herein as the percentage of amino acid residues in the candidate sequence that are identical with the parent antibody residues, after aligning the sequences and introducing gaps, if necessary, to achieve the maximum percent sequence identity. None of N-terminal, C-terminal, or internal extensions, deletions, or insertions into the antibody sequence shall be construed as affecting sequence identity or homology. The variant retains the ability to bind the receptor and preferably has properties that are superior to those of the parent antibody. For example, the variant may have a stronger binding affinity, enhanced ability to activate the receptor, etc. The variant antibody of particular interest herein is one that displays at least about the same level of enhancement in biological activity when compared to the parent antibody.

The “parent” antibody herein is one that is encoded by an amino acid sequence used for the preparation of the variant. Preferably, the parent antibody has a human framework region and has human antibody constant region(s). For example, the parent antibody may be a humanized or human antibody.

An “isolated” antibody is one that has been identified and separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In preferred embodiments, the antibody will be purified to greater than 95% by weight of antibody as determined by the Bradford method, and most preferably more than 99% by weight, to a degree sufficient to obtain at least 15 residues of N-terminal or internal amino acid sequence by use of a spinning cup sequenator, or to homogeneity by SDS-PAGE under reducing or non-reducing conditions using Coomassie Blue or, preferably, silver stain. Isolated antibody includes the antibody in situ within recombinant cells since at least one component of the antibody's natural environment will not be present. Ordinarily, however, isolated antibody will be prepared by at least one purification step.

The term “epitope tagged” when used herein refers to an antibody fused to an “epitope tag”. The epitope tag polypeptide has enough residues to provide an epitope against which an antibody can be made, yet is short enough such that it does not interfere with the activity of the antibody. The epitope tag preferably is sufficiently unique so that the antibody recognizing it does not substantially cross-react with other epitopes. Suitable tag polypeptides generally have at least 6 amino acid residues and usually between about 8-50 amino acid residues (preferably between about 9-30 residues). Examples include the flu HA tag polypeptide and its antibody 12CA5 (Field et al., Mol. Cell. Biol., 8: 2159-2165, 1988); the c-myc tag and the 8F9, 3C7, 6EI0, G4, 137 and 9E10 antibodies thereto (Evan et al., Mol. Cell. Biol., 5: 3610-3616, 1985); and the Herpes Simplex Virus glycoprotein D (gD) tag and its antibody (Paborsky et al., Prot. Eng., 3: 547-553, 1990). In certain embodiments, the epitope tag is a “salvage receptor binding epitope”. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

“Affinity matured antibodies” are antibodies that have had their binding affinity and/or biological activity increased by altering the type or location of one or more residues in the variable region. An example of alteration is a mutation that may be in either a CDR or a framework region. An affinity matured antibody will typically have its binding affinity increased above that of the isolated or natural antibody or fragment thereof by from 2- to 500-fold. Preferred affinity matured antibodies will have nanomolar or even picomolar affinities to the receptor antigen. Affinity matured antibodies are produced by procedures known in the art, such as VH and VL domain shuffling, mutagenesis of CDR and/or framework residues, etc. (see, e.g., Marks et al., BioTechnology, 10: 779-783, 1992; Barbas et al., Proc. Nat. Acad. Sci. USA, 91: 3809-3813, 1994; Schier et al., Gene, 169: 147-155, 1995; Yelton et al., J. Immunol., 155: 1994-2004, 1995; Jackson et al., J. Immunol., 154: 3310-19, 1995; Hawkins et al., J. Mol. Biol., 226: 889-896, 1992).

### Immunoglobulin Fusion Protein and Derivative Construct

In certain embodiments, the construct is created by fusing (joining) an immunoglobulin family protein modified to include the heterologous binding sequence to an amino acid sequence of another protein (or portion thereof, preferably an at least 10, 20, or 50 amino acid portion thereof) that is not the modified immunoglobulin, thereby creating a fusion (or chimeric) construct. Preferably, the fusion is via covalent bond (for example, but not by way of limitation, a peptide bond) at either the N-terminus or the C-terminus.

The construct may be further modified, e.g., by the covalent attachment of any type of molecule, as long as such covalent attachment does not prevent or inhibit specific binding of the sythebody to its target antigen. For example, but not by way of limitation, the construct may be further modified, e.g., by glycosylation, acetylation, PEGylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc.

In specific embodiments of the invention, the construct is covalently linked to a therapeutic molecule, for example, to target the therapeutic molecule to a particular cell type or tissue, e.g., an accessory or antigen-presenting cell. The therapeutic molecule can be any type of therapeutic molecule known in the art, for example, but not limited to, a chemotherapeutic agent, a toxin, such as ricin, an antisense oligonucleotide, a radionuclide, an antibiotic, anti-viral, or anti-parasitic, etc.

### Methods of Producing the Constructs

Constructs can be produced by any method known in the art for the synthesis of immunoglobulins, in particular, by chemical synthesis or by recombinant expression, and are preferably produced by recombinant expression techniques.

Recombinant expression of constructs requires construction of a nucleic acid encoding the construct. Such an isolated nucleic acid that contains a nucleotide sequence encoding the construct can be produced using any method known in the art.

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization, B. D. Hames & S. J. Higgins eds. (1985); Transcription And Translation, B. D. Hames & S. J. Higgins, eds. (1984); Animal Cell Culture, R. I. Freshney, ed. (1986); Immobilized Cells And Enzymes, IRL Press, (1986); B. Perbal, A Practical Guide To Molecular Cloning (1984); F. M. Ausubel et al. (eds.), Current Protocols in Molecular Biology, John Wiley & Sons, Inc. (1994).

### Construct Nucleic Acids

Accordingly, the invention provides nucleic acids that contain a nucleotide sequence encoding a construct of the invention.

A nucleic acid that encodes a construct may be assembled from chemically synthesized oligonucleotides (e.g., as described in Kutmeier et al., BioTechniques, 1994, 17:242), that briefly, involves the synthesis of a set of overlapping oligonucleotides containing portions of the sequence encoding the protein, annealing and ligation of those oligonucleotides, and then amplification of the ligated oligonucleotides by PCR.

Accordingly, the invention provides a method of producing a nucleic acid encoding a construct, the method comprising: (a) synthesizing a set of oligonucleotides, the set comprising oligonucleotides containing a portion of the nucleotide sequence that encodes the construct and oligonucleotides containing a portion of the nucleotide sequence that is complementary to the nucleotide sequence that encodes the construct, and each of the oligonucleotides having overlapping terminal sequences with another oligonucleotide of the set, except for those oligonucleotides containing the nucleotide sequences encoding the N-terminal and C-terminal portions of the synthetic synthebody; (b) allowing the oligonucleotides to hybridize or anneal to each other; and (c) ligating the hybridized oligonucleotides, such that a nucleic acid containing the nucleotide sequence encoding the synthetic synthebody is produced.

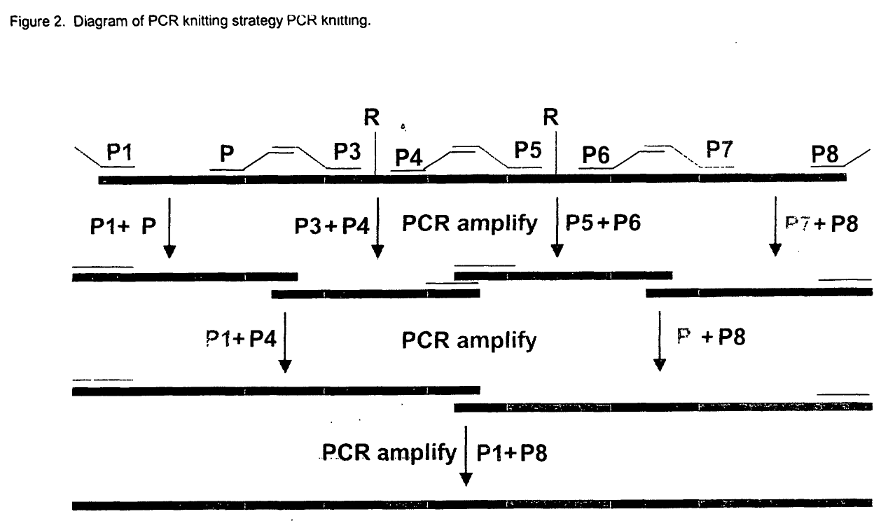

Another method for producing a nucleic acid encoding a construct is to modify nucleic acid sequences that encode an immnunoglobulin superfamily molecule, e.g., an antibody molecule or at least the variable region thereof, using the “PCR knitting” approach (FIG. 2). In “PCR knitting”, nucleic acid sequences, such as the consensus variable region sequences shown in Example 1, are used as templates for a series of PCR reactions that result in the selective insertion of a nucleotide sequence that encodes the desired peptide sequence (in this example, the TPO receptor binding sequence of TPO) into one or more CDRs of the variable domain. Oligonucleotide primers are designed for these PCR reactions that contain regions complementary to the framework sequences flanking the designated CDR at the 3′ ends and sequences that encode the peptide sequence to be inserted at the 5′ends. In addition, these oligonucleotides contain approximately ten bases of complementary sequences at their 5′ends. These oligonucleotide primers can be used with additional flanking primers to insert the desired nucleotide sequence into the selected CDR as shown in FIG. 2 resulting in the production of a nucleic acid coding for the synthebody.

Alternatively, a nucleic acid containing a nucleotide sequence encoding a construct can be constructed from a nucleic acid containing a nucleotide sequence encoding, e.g., an antibody molecule, or at least a variable region of an antibody molecule. Nucleic acids containing nucleotide sequences encoding antibody molecules can be obtained either from existing clones of antibody molecules or variable domains or by isolating a nucleic acid encoding an antibody molecule or variable domain from a suitable source, preferably a cDNA library, e.g., an antibody DNA library or a cDNA library prepared from cells or tissue expressing a repertoire of antibody molecules or a synthetic antibody library (see, e.g., Clackson et al., Nature, 1991, 352:624; Hane et al., Proc. Natl. Acad. Sci. USA, 1997, 94:4937), for example, by hybridization using a probe specific for the particular antibody molecule or by PCR amplification using synthetic primers hybridizable to the 3′ and 5′ ends of the sequence.

If a convenient restriction enzyme site is available in the nucleotide sequence of the CDR, then the sequence can be cleaved with the restriction enzyme and a nucleic acid fragment containing the nucleotide sequence encoding the binding site can be ligated into the restriction site. The nucleic acid fragment containing the binding site can be obtained either from a nucleic acid encoding all or a portion of the protein containing the binding site or can be generated from synthetic oligonucleotides containing the sequence encoding the binding site and its reverse complement.

The nucleic acid encoding the modified antibody optionally contains a nucleotide sequence encoding a leader sequence that directs the secretion of the synthebody molecule.

### Construct Expression

Once a nucleic acid encoding a construct is obtained, it may be expressed or it may be introduced into a vector containing the nucleotide sequence encoding the constant region of the antibody (see, e.g., PCT Publications WO 86/05807 and WO 89/01036; and U.S. Pat. No. 5,122,464). Vectors containing the complete light or heavy chain for co-expression are available to allow the expression of a complete antibody molecule and are known in the art, for example, pMRRO10.1 and pGammal (see also, Bebbington, Methods a companion to Methods in Enzymology, 1991, 2:136-145).

The expression vector can then be transferred to a host cell in vitro or in vivo by conventional techniques and the transfected cells can be cultured by conventional techniques to produce a construct of the invention. Specifically, once a variable region of the modified antibody has been generated, the modified antibody can be expressed, for example, by the method exemplified in the Examples (see also Bebbington, supra). For example, by transient transfection of the expression vector encoding a construct into COS cells, culturing the cells for an appropriate period of time to permit construct expression, and then taking the supernatant from the COS cells, which supernatant contains the secreted, expressed synthebody.

The host cells used to express the recombinant construct of the invention may be either bacterial cells such as Escherichia coli, particularly for the expression of recombinant antibody fragments or, preferably, eukaryotic cells, particularly for the expression of recombinant immunoglobulin molecules. In particular, mammalian cells such as Chinese hamster ovary cells (CHO) or COS cells, used in conjunction with a vector in which expression of the construct is under control of the major intermediate early gene promoter element from human cytomegalovirus, is an effective expression system for immunoglobulins (Foecking et al., Gene, 1986, 45:101; Cockett et al., BioTechnology, 1990, 8:662).

A variety of host-expression vector systems may be utilized to express the construct coding sequences of the invention. Such host-expression systems represent vehicles by which the coding sequences of interest may be produced and subsequently purified, but may also be used to transform or transfect cells with the appropriate nucleotide coding and control sequences to produce the antibody product of the invention in situ. These systems include, but are not limited to, microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing the antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; mammalian cell systems (e.g., COS, CHO, BHK, 293, and 3T3 cells) harboring recombinant expression constructs containing promoters derived from the genome of mammalian cells (e.g., the metallothionein promoter) or from mammalian viruses (e.g., the adenovirus late promoter; the vaccinia virus 7.5K promoter); and transgenic animal systems, particularly for expression in milk (e.g., U.S. Pat. Nos. 5,831,141 and 5,849,992, which describe transgenic production of antibodies in milk; U.S. Pat. No. 4,873,316).

Expression of the construct may be controlled by any promoter/enhancer element known in the art, but these regulatory elements must be functional in the host selected for expression. Promoters that may be used to control gene expression include, but are not limited to, cytomegalovirus (CMV) promoter (U.S. Pat. Nos. 5,385,839 and 5,168,062), the SV40 early promoter region (Benoist and Chambon, Nature, 1981, 290:304-310), the promoter contained in the 3′ long terminal repeat of Rous sarcoma virus (Yamamoto, et al., Cell, 1980, 22:787-797), the herpes thymidine kinase promoter (Wagner et al., Proc. Natl. Acad. Sci. U.S.A., 1981, 25 78:1441-1445), the regulatory sequences of the metallothionein gene (Brinster et al., Nature, 1982, 296:39-42); prokaryotic expression vectors such as the β-lactamase promoter (Villa-Komaroff, et al., Proc. Natl. Acad. Sci. U.S.A., 1978, 75:3727-3731), or the tac promoter (DeBoer, et al., Proc. Natl. Acad. Sci. U.S.A., 1983, 80:21-25); see also “Useful proteins from recombinant bacteria” in Scientific American, 1980, 30 242:74-94; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al, Nature, 1985, 315:338-340; Kollias et at, 1986, Cell 46:89-94), hematopoietic stem cell differentiation factor promoters, erythropoietin receptor promoter (Maouche et al., Blood, 1991, 15:2557), etc.

In bacterial systems, a number of expression vectors may be advantageously selected depending upon the use intended for the construct being expressed. For example, when a large quantity of such a protein is to be produced, for the generation of pharmaceutical compositions of a construct, vectors which direct the expression of high levels of fusion protein products that are readily purified may be desirable. Such vectors include, but are not limited to, the E. coli expression vector pUR278 (Ruther et at, EMBO J., 1983, 2:1791), in which the coding sequence may be ligated individually into the vector in frame with the lac Z coding region so that a fusion protein is produced; pIN vectors (Inouye & Inouye, Nucleic Acids Res., 1985, 13:3101-3109; Van Hleeke & Schuster, J. Biol. Chem., 1989, 264:5503-5509); and the like. pGEX vectors may also be used to express foreign polypeptides as fusion proteins with glutathione S-transferase (GST). In general, such fusion proteins are soluble and can easily be purified from lysed cells by adsorption and binding to a matrix of glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example, the polyhedrin gene) of the virus and placed under control of an AcNPV promoter (for example, the polyhedrin promoter).

In mammalian host cells, a number of viral-based and non-viral-based expression systems may be utilized. In cases where an adenovirus is used as an expression vector, the coding sequence of interest may be ligated to an adenovirus transcription/translation control complex, e.g., the late promoter and tripartite leader sequence. This chimeric gene may then be inserted into the adenovirus genome. Insertion in a non-essential region of the viral genome (e.g., region E1 or E3) will result in a recombinant virus that is viable and capable of expressing the construct in infected hosts (see, e.g., Logan and Shenk, Proc. Natl. Acad. Sci. U.S.A., 1984, 81:3655-3659). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol., 1987, 153:516-544).

Additionally, a host cell strain may be chosen that modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138.

For long-term, high-yield production of recombinant proteins, stable expression is preferred. For example, cell lines that stably express the antibody may be engineered. Rather than using expression vectors that contain viral origins of replication, host cells can be transformed with DNA controlled by appropriate expression control elements (e.g., promoter, enhancer sequences, transcription terminators, polyadenylation sites, etc.) and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which, in turn, can be cloned and expanded into cell lines. This method may advantageously be used to engineer cell lines that express the antibody. Such engineered cell lines may be particularly useful in screening and evaluation of compounds that interact directly or indirectly with the antibody.

A number of selection systems may be used, including but not limited to the herpes simplex virus thyrmidine kinase (Wigler et al., Cell, 1977, 11:223), hypoxanthine-guanine phosphoribosyltransferase (Szybalska and Szybalski, Proc. Natl. Acad. Sci. USA, 1962, 48:2026), and adenine phosphoribosyltransferase (Lowy et al., Cell, 1980, 22:817) genes can be employed in tk-, hgprt-, or aprt-cells, respectively. Also, antimetabolite resistance can be used as the basis of selection for the following genes: dhfr, which confers resistance to methotrexate (Wigler et al., Proc. Natl. Acad. Sci. USA, 1980, 77:3567; O'Hare et al., Proc. Natl. Acad. Sci. USA, 1981, 78:1527); gpt, which confers resistance to mycophenolic acid (Mulligan and Berg, Proc. Natl. Acad. Sci. USA, 1981, 78:2072); neo, which confers resistance to the aminoglycoside G-418 (Colberre-Garapin et al., J. Mol. Biol., 1981, 150:1); and hygro, which confers resistance to hygromycin (Santerre et al., Gene, 1984, 30:147).

The expression levels of the construct can be increased by vector amplification (for a review, see Bebbington and Hentschel, The Use of Vectors Based on Gene Ampliflication for the Expression of Cloned Genes in Mammalian Cells in DNA Cloning, Vol. 3, Academic Press, New York, 1987). When a marker in the vector system expressing a construct is amplifiable, increases in the level of inhibitor present in the culture medium of the host cell will increase the number of copies of the marker gene. Since the amplified region is associated with the construct gene, production of the construct will also increase (Crouse et al., Mol. Cell. Biol., 1983, 3:257).

In a specific embodiment in which the construct is an antibody (immunoglobulin), the host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used that encodes both heavy and light chain polypeptides. In such situations, the light chain should be placed before the heavy chain to avoid an excess of toxic free heavy chain (Proudfoot, Nature, 1986, 322:562; Kohler, Proc. Natl. Acad Sci. USA, 1980, 77:2197). The coding sequences for the heavy and light chains may comprise cDNA or genomic DNA.

The invention provides a recombinant cell that contains a vector which encodes a synthetic antibody that has a CDR that contains the amino acid sequence of an active binding site from a member of a binding pair.

### Viral and Non-Viral Vectors

Preferred vectors, particularly for cellular assays in vitro and in vivo, are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide domain fragment thereof can be introduced in vivo, ex vivo, or in vitro using a viral vector or through direct introduction of DNA. Expression in targeted tissues can be affected by targeting the transgenic vector to specific cells, such as with a viral vector or a receptor ligand, or by using a tissue-specific promoter, or both. Targeted gene delivery is described in PCT Publication No. WO 95/28494.

Viral vectors commonly used for in vivo or ex vivo targeting and therapy procedures are DNA-based vectors and retroviral vectors. Methods for constructing and using viral vectors are known in the art (see, e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome that are necessary for encapsidating the genome to produce viral particles.

DNA viral vectors include an attenuated or defective DNA virus, such as but not limited to, herpes simplex virus (HSV), papillomavirus, Epstein Barr virus (EBV), adenovirus, adeno-associated virus (AAV), and the like. Defective viruses that entirely or almost entirely lack viral genes are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci., 1991, 2:320-330), defective herpes virus vector lacking a glyco-protein L gene, or other defective herpes virus vectors (PCT Publication Nos. WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996).

Various companies produce viral vectors commercially, including, but not limited to, Avigen, Inc. (Alameda, Calif.; AAV vectors), Cell Genesys (Foster City, Calif.; retroviral, adenoviral, AAV, and lentiviral vectors), Clontech (retroviral and baculoviral vectors), Genovo, Inc. (Sharon Hill, Pa.; adenoviral and AAV vectors), Genvec (France; adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenovirus vectors. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to using type 2 or type 5 human adenoviruses (Ad 2 or Ad 5) or adenoviruses of animal origin (see PCT Publication No. WO 94/26914). Those adenoviruses of animal origin that can be used within the scope of the present invention include adenoviruses of canine, bovine, murine (example: Mav1, Beard et al., Virology, 1990, 75-81), ovine, porcine, avian, and simian (example: SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (PCT Publication Nos. WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (Levrero et al., Gene, 1991, 101:195; European Publication No. EP 185 573; Graham, EMBO J., 1984, 3:2917; Graham et al., J. Gen. Virol., 1977, 36:59). Recombinant adenoviruses are recovered and purified using standard molecular biological techniques that are well known to one of ordinary skill in the art.

Adeno-associated viruses. The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described (see, PCT Publication Nos. WO 91/18088 and WO 93/09239; U.S. Pat. Nos. 4,797,368 and 5,139,941; European Publication No. EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

Retrovirus vectors. In another embodiment the gene can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol., 1988, 62:1120; U.S. Pat. No. 5,124,263; European Publication Nos. EP 453 242 and EP178 220; Bernstein et al, Genet. Eng.,1985, 7:235; McCormick, BioTechnology, 1985, 3:689; PCT Publication No. WO 95/07358; and Kuo et al., Blood, 1993, 82:845. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukemia virus”) MSV (“murine Moloney sarcoma virus”), HaSV (“Harvey sarcoma virus”); SNV (“spleen necrosis virus”); RSV (“Rous sarcoma virus”) and Friend virus. Suitable packaging cell lines have been described in the prior art, in particular the cell line PA317 (U.S. Pat. No. 4,861,719); the PsiCRIP cell line (PCT Publication No. WO 90/02806) and the GP+envAm-12 cell line (PCT Publication No. WO 89/07150). In addition, the recombinant retroviral vectors can contain modifications within the LTRs for suppressing transcriptional activity as well as extensive encapsidation sequences that may include a part of the gag gene (Bender et al., J. Virol., 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permit one cycle of retroviral replication and amplifies transfection efficiency (see PCT Publication Nos. WO 95/22617, WO 95/26411, WO 96/39036 and WO 97/19182).

Lentivirus vectors. In another embodiment, lentiviral vectors can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. For a review, see, Naldini, Curr. Opin. Biotechnol., 1998, 9:457-63; see also Zufferey, et al., J. Virol., 1998, 72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 10⁶ IU/ml for at least 3 to 4 days (Kafri, et al., J. Virol., 1999, 73: 576-584). The vector produced by the inducible cell line can be concentrated as needed for efficiently transducing non-dividing cells in vitro and in vivo.

Non-viral vectors. In another embodiment, the vector can be introduced in vivo by lipofection, as naked DNA, or with other transfection facilitating agents (peptides, polymers, etc.). Synthetic cationic lipids can be used to prepare liposomes for in vivo. transfection of a gene encoding a marker (Felgner, et. al., Proc. Natl. Acad. Sci. U.S.A., 1987, 84:7413-7417; Felgner and Ringold, Science, 1989, 337:387-388; see Mackey, et al., Proc. Natl. Acad. Sci. U.S.A., 1988, 85:8027-8031; Ulmer et al., Science, 1993, 259:1745-1748). Useful lipid compounds and compositions for transfer of nucleic acids are described in PCT Patent Publication Nos. WO 95/18863 and WO 96/17823, and in U.S. Pat. No. 5,459,127. Lipids may be chemically coupled to other molecules for the purpose of targeting (see Mackey, et al., supra). Targeted peptides, e.g., hormones or neurotransmitters, and proteins such as antibodies, or non-peptide molecules could be coupled to liposomes chemically.

Other molecules are also useful for facilitating transfection of a nucleic acid in vivo, such as a cationic oligopeptide (e.g., PCT Patent Publication No. WO 95/21931), peptides derived from DNA binding proteins (e.g., PCT Patent Publication No. WO 96/25508), or a cationic polymer (e.g., PCT Patent Publication No. WO 95/21931).

It is also possible to introduce the vector in vivo as a naked DNA plasmid. Naked DNA vectors for gene therapy can be introduced into the desired host cells by methods known in the art, e.g., electroporation, microinjection, cell fusion, DEAE dextran, calcium phosphate precipitation, use of a gene gun, or use of a DNA vector transporter (see, e.g., Wu et al., J. Biol. Chem., 1992, 267:963-967; Wu and Wu, J. Biol. Chem., 1988, 263:14621-14624; Canadian Patent Application No. 2,012,311; Williams et al., Proc. Natl. Acad. Sci. USA, 1991, 88:2726-2730). Receptor-mediated DNA delivery approaches can also be used (Curiel et al., Hum. Gene Ther., 1992, 3:147-154; Wu and Wu, J. Biol. Chem., 1987, 262:4429-4432). U.S. Pat. Nos. 5,580,859 and 5,589,466 disclose delivery of exogenous DNA sequences, free of transfection facilitating agents, in a mammal. Recently, a relatively low voltage, high efficiency in vivo DNA transfer technique, termed electrotransfer, has been described (Mir et al., C.P. Acad. Sci., 1988, 321:893; PCT Publication Nos. WO 99/01157; WO 99/01158; WO 99/01175).

### PROTEIN CONSTRUCTION PURIFICATION

A suitable method for purifying protein constructs of the invention comprises contacting a source containing the protein construct molecules with an immobilized receptor polypeptide, specifically MPL or a MPL fusion polypeptide, under conditions whereby the protein construct molecules to be purified are selectively adsorbed onto the immobilized receptor polypeptide, washing the immobilized support to remove non-adsorbed material, and eluting the molecules to be purified from the immobilized receptor polypeptide with an elution buffer. The source containing the construct may be a recombinant cell culture where the concentration of antibody in either the culture medium or in cell lysates is generally higher than in plasma or other natural sources. In addition to MPL affinity purification, the constructs of the invention may be purified using conventional biochemical methods comprising: removing particulate debris (either host cells or lysed fragments) by, e.g., centrifugation or ultrafiltration (optionally, protein may be concentrated with a commercially available protein concentration filter), followed by separating the antibody from other impurities by one or more steps selected from immunoaffinity, ion-exchange (e.g., DEAE or matrices containing carboxymethyl or sulfopropyl groups), Blue-SEPHAROSE, CM Blue-SEPHAROSE, MONO-Q, MONO-S, lentil lectin-SEPHAROSE, WGA-SEPHAROSE, Con A-SEPHAROSE, Ether TOYPEARL, Butyl TOYPEARL, Phenyl TOYPEARL, protein A SEPHAROSE, SDS-PAGE, reverse phase HPLC (e.g., silica gel with appended aliphatic groups) or SEPHADEX molecular sieve or size exclusion chromatography (optionally, followed by ethanol or ammonium sulfate precipitation). Protease inhibitors (e.g., phenylmethylsulfonylfluoride [PMSF], leupeptin, peptstatin A) may be included in any of the foregoing steps to inhibit proteolysis.

### Therapeutic Use of Constructs

The invention also provides methods for treating or preventing diseases and disorders by administration of therapeutics of the invention. Such therapeutics include the constructs of the invention and nucleic acids encoding the constructs of the invention.

Generally, administration of products of a species origin or species reactivity that is the same species as that of the subject is preferred. Thus, in administration to humans, the therapeutic methods of the invention use a synthebody that is derived from a human antibody; in other embodiments, the methods of the invention use a modified antibody that is derived from a chimeric or humanized antibody.

The method of the invention includes administering to a subject in need of such treatment or prevention a therapeutic of the invention, i.e., a synthebody, that specifically binds to TPO receptor, which synthebody comprises a variable domain with a CDR containing the amino acid sequence capable of efficient TPO receptor binding (preferably, monomeric or dimeric sequence IEGPTLRQWLAARA or its derivatives), or a nucleic acid vector encoding such synthebody.

Pharmaceutical compositions containing the synthebodies of the invention that specifically bind TPO receptor or nucleic acids encoding such synthebodies can be used in the treatment or prevention of diseases or disorders associated with the function of this receptor. Specifically, in embodiments discussed in more detail in the subsections that follow, TPO receptor agonist synthebodies and nucleic acids of the present invention can be used to treat various hematopoietic or immune disorders. Most importantly, these synthebodies can be used to treat or prevent thrombocytopenia in mammals (including humans) suffering from thrombocytopenia-associated bone marrow hypoplasia following chemotherapy, radiation therapy or bone marrow transfusion; disseminated intravascular coagulation (DIC); immune thrombocytopenia (including HIV-induced ITP and non-HIV-induced ITP); chronic idiopathic thrombocytopenia; congenital thrombocytopenia; myelodysplasia, and thrombotic thrombocytopenia. In addition, they can be used for the mobilization, amplification and ex vivo expansion of stem cells and committed precursor cells for autologous and allogeneic transplantation as well as for the expansion of stem cells destined for gene therapy (e.g., retroviral vector-based).

The invention further includes a method for stimulating proliferation and/or differentiation and/or growth and/or modulating apoptosis of a hematopoietic cell (e.g., megakaryocyte, CD34+ cell, granulocytic macrophage progenitor, and erythroid progenitor), comprising contacting such cell with an effective amount of the synthebody of the invention. Optionally, the synthebody may be used in combination with at least one other protein or peptide having hematopoietic activity (e.g., SCF, IL-1, IL-3, IL-6, IL-11, LIF, G-CSF, GM-CSF, M-CSF, EPO, kit ligand, and-interferon). As disclosed herein, this method is employed for the mobilization, amplification and ex vivo expansion of hematopoietic stem cells or committed hematopoietic precursor cells and is useful for autologous or allogeneic transplantation or for the expansion of stem cells destined for gene therapy (e.g., retrovirus vector-based). Thus, in a specific embodiment, the invention provides an ex vivo method of treating thrombocytopenia in a mammal (e.g., human) comprising: (i) obtaining a population of megakaryocyte precursor cells from the subject to be treated; (ii) treating said cells with the TPO receptor agonist synthebody of the invention; and (iii) administering said treated cells to said subject, to increase the number of megakaryocytes present in said subject.

In another embodiment, the invention provides a method for stimulating megakaryocytopoietic or thrombopoietic activity in a subject, which method comprises administering to said subject a pharmaceutical composition comprising the TPO agonist synthebody. Optionally, the synthebody may be used in combination with at least one other protein or peptide having megakaryocytopoietic or thrombopoietic activity (e.g., SCF, IL-1, IL-2, IL-3, IL-5, IL-6, IL-7, IL-8, IL-9, IL-11, LIF, G-CSF, GM-CSF, M-CSF, EPO, kit ligand, -interferon, IGF-1, and lymphotoxin [LT]). This method can be used to treat or prevent thrombocytopenia in mammals (including humans) suffering from thrombocytopenia-associated bone marrow hypoplasia following chemotherapy, radiation therapy or bone marrow transfusion; disseminated intravascular coagulation (DIC); immune thrombocytopenia (including HIV-induced ITP and non-HIV-induced ITP); chronic idiopathic thrombocytopenia; congenital thrombocytopenia; myelodysplasia, and thrombotic thrombocytopenia. In a preferred embodiment, the synthebody of the invention is used to treat or prevent thrombocytopenia resulting from chemotherapy, radiation therapy or bone marrow transfusion. In this embodiment, the synthebody can be optionally administered prior to chemotherapy, radiation therapy, or bone marrow transplantation.

The subjects to which the present invention is applicable may be any mammalian or vertebrate species, which include, but are not limited to, cows, horses, sheep, pigs, fowl (e.g., chickens), goats, cats. dogs, hamsters, mice, rats, monkeys, rabbits, chimpanzees, and humans. In a preferred embodiment, the subject is a human.

### Diagnostic and Research Applications

The synthebodies of the invention are useful in vitro as unique tools for understanding the biological role of TPO and its receptor c-MPL, including the evaluation of the many factors thought to influence, and be influenced by, the production of TPO and the receptor binding process. The present synthebodies are also useful in the development of other compounds that bind to and activate the TPO receptor, because the present synthebodies provide important information on the relationship between structure and activity that should facilitate such development.

The synthebodies of the invention are also useful as competitive binders in assays to screen for new TPO receptor agonists. In competition assays, the synthebodies of the invention can be used without modification or can be modified in a variety of ways, for example, by labeling, such as covalently or non-covalently joining a moiety that directly or indirectly provides a detectable signal. Possibilities for direct labeling include without limitation: radiolabels (e.g., 1251), enzymes (e.g., peroxidase and alkaline phosphatase; see also U.S. Pat. No. 3,645,090), and fluorescent labels (see, e.g., U.S. Pat. No. 3,940,475). Possibilities for indirect labeling include, e.g., biotinylation of one constituent followed by binding to avidin coupled to one of the above labeled groups.

Based on their ability to bind to the TPO receptor, the synthebodies of the present invention can be used as reagents for detecting TPO receptors on living cells, fixed cells, in biological fluids, in tissue homogenates, in purified, natural biological materials, etc. Binding of antibodies to the TPO receptors may be detected using direct labeling of synthebodies or immunochemical methods such as Western blotting, ELISA, etc.

The synthebodies of the present invention can be also used in MPL receptor purification, or in purifying cells expressing TPO receptors on their surface (see above).

The synthebodies of the present invention can be further utilized as commercial reagents for various medical research and diagnostic uses. Such uses include but are not limited to: (1) use as a calibration standard for quantitating the activities of candidate TPO agonists in a variety of functional assays; (2) use to maintain the proliferation and growth of TPO-dependent cell lines; (3) use in structural analysis of the TPO-receptor through co-crystallization; (4) use to investigate the mechanism of TPO signal transduction/receptor activation; and (5) other research and diagnostic applications wherein the TPO-receptor is preferably activated or such activation is conveniently calibrated against a known quantity of a TPO agonist, and the like.

The synthebodies of the present invention can be used for the in vitro expansion of hematopoietic progenitor cells (in particular, megakaryocytes and their committed progenitors), both in conjunction with additional cytokines or on their own (see, e.g., PCT Publication No. WO 95/05843, which is incorporated herein by reference). As disclosed herein, amelioration of the thrombocytopenia by the synthebodies of the present invention can be hastened by infusing patients post-chemotherapy or radiation therapy with a population of his or her own cells enriched for megakaryocytes and immature precursors produced in in vitro culture.

Synthebodies of the present invention can be also used for the mobilization, amplification and ex vivo expansion of non-megakaryocytic stem cells and committed precursor cells for autologous and allogeneic transplantation as well as for the expansion of stem cells destined for gene therapy (e.g., retroviral vector-based).

### Treatment and Prevention of Thrombocytopenia

The TPO receptor agonist synthebodies of the invention can be administered to warm blooded animals, including humans, to stimulate in vivo TPO receptor-mediated proliferation and/or differentiation and/or modulate apoptosis. Thus, the present invention encompasses methods for therapeutic treatment of TPO-related disorders that comprise administering a synthebody of the invention or its fragment or derivative in amounts sufficient to mimic the effect of TPO on TPO receptor in vivo. Such synthebody may be used in a sterile pharmaceutical preparation or formulation to stimulate hematopoietic (preferably megakaryocytopoietic or thrombopoietic) activity in patients suffering from thrombocytopenia-associated bone marrow hypoplasia (e.g., aplastic anemia following chemotherapy or radiation therapy for treatment of leukemia or solid tumors, or myeloablative chemotherapy for autologous or allogeneic bone marrow transplant), disseminated intravascular coagulation (DIC), immune thrombocytopenia (including HIV-induced ITP and non HIV-induced ITP), chronic idiopathic thrombocytopenia, congenital thrombocytopenia, myelodysplasia, and thrombotic thrombocytopenia.

According to one embodiment, the synthebodies of the present invention are useful for treating thrombocytopenia associated with bone marrow transfusions, radiation therapy, or chemotherapy. The compounds typically will be administered prophylactically prior to chemotherapy, radiation therapy, or bone marrow transplant or after such exposure. In addition, the synthebodies of the present invention can be used for the mobilization, amplification and ex vivo expansion of stem cells and committed precursor cells for autologous and allogeneic transplantation as well as for the expansion of stem cells destined for gene therapy.

The TPO receptor agonist antibody of the instant invention may be used in the same way and for the same indications as thrombopoietin (TPO). Some forms of the antibody have a longer half-life than native, recombinant “native-like” or pegylated TPO and thus are used in cases where a longer half-life is beneficial.

Accordingly, the present invention also provides pharmaceutical compositions comprising, as an active ingredient, at least one of the synthebodies of the invention or nucleic acids encoding such antibodies in association with a pharmaceutical carrier or diluent.

The biologically active TPO receptor agonist antibody of the present invention may be employed alone or in combination with other cytokines, hematopoietins, interleukins, growth factors, or antibodies in the treatment of the above-identified disorders and conditions. Thus, the presentinstant compounds may be employed in combination with other protein or peptide having hematopoietic activity including (but not limited to) stem cell factor (SCF), IL-1, IL-3, IL-6, IL-11, leukaemia inhibiting factor (LIF), G-CSF, GM-CSF, M-CSF, erythropoietin (EPO), kit ligand, and -interferon.

In some embodiments of the invention, TPO antagonists are preferably first administered to patients undergoing chemotherapy or radiation therapy followed by administration of the TPO agonists of the invention. The activity of the synthebodies of the present invention can be evaluated either in vitro or in vivo in one of the numerous models described by McDonald (Am. J. Ped. Hematol./Oncol., 14:8-21, 1992).

When used for in vivo administration, the antibody formulation must be sterile. This is readily accomplished by filtration through sterile filtration membranes, prior to or following lyophilization and reconstitution. The antibody ordinarily will be stored in lyophilized form or in solution. Therapeutic antibody compositions generally are placed into a container having a sterile access port, for example, an intravenous solution bag or vial having a stopper pierceable by a hypodermic injection needle. The route of antibody administration is in accord with known methods, e.g., injection or infusion by intravenous, intraperitoneal, intracerebral, intramuscular, intraocular, intraarterial, intrathecal, inhalation or intralesional routes, or by sustained release systems as noted below. The antibody is preferably administered continuously by infusion or by bolus injection.

An effective amount of antibody to be employed therapeutically will depend, for example, upon the therapeutic objectives, the route of administration, and the condition of the patient. Accordingly, it will be necessary for the therapist to titer the dosage and modify the route of administration as required to obtain the optimal therapeutic effect. Typically, the clinician will administer antibody until a dosage is reached that achieves the desired effect. The progress of this therapy is easily monitored by conventional assays.

### Gene Therapy

In a specific embodiment, expression vectors comprising a sequence encoding a synthebody of the invention are administered to treat or prevent the hematopoietic disorders described above.

In this embodiment of the invention, the therapeutic vector encodes a sequence that produces intracellularly (without a leader sequence) or extracellularly (with a leader sequence) a synthebody of the invention.

Any of the methods for gene therapy available in the art can be used according to the present invention. Exemplary methods are described below.

For general reviews of the methods of gene therapy, see, Goldspiel et al., Clinical Pharmacy, 1993, 12:488-505; Wu and Wu, Biotherapy, 1991, 3:87-95; Tolstoshev, Ann. Rev. Pharmacol. Toxicol., 1993, 32:573-596; Mulligan, Science, 1993, 260:926-932; and Morgan and Anderson, Ann. Rev. Biochem., 1993, 62:191-217; May, TIBTECH, 1993, 11:155-215). Methods commonly known in the art of recombinant DNA technology that can be used are described in Ausubel et al., (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al., (eds.), 1994, Current Protocols in Human Genetics, John Wiley & Sons, NY. Vectors suitable for gene therapy are described above.

In one aspect, the therapeutic vector comprises a nucleic acid that expresses the synthebody in a suitable host. In particular, such a vector has a promoter operationally linked to the coding sequence for the synthebody. The promoter can be inducible or constitutive and, optionally, tissue-specific. In another embodiment, a nucleic acid molecule is used in which the antibody coding sequences and any other desired sequences are flanked by regions that promote homologous recombination at a desired site in the genome, thus providing for intrachromosomal expression of the synthebody (Koller and Smithies, Proc. Natl. Acad. Sci. USA, 1989, 86:8932-8935; Zijlstra et al., Nature, 1989, 342:435-438).

Delivery of the vector into a patient may be either direct, in which case the patient is directly exposed to the vector or a delivery complex, or indirect, in which case, cells are first transformed with the vector in vitro then transplanted into the patient. These two approaches are known, respectively, as in vivo and ex vivo gene therapy.

In a specific embodiment, the vector is directly administered in vivo, where it enters the cells of the organism and mediates expression of the antibodies. This can be accomplished by any of numerous methods known in the art, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell-surface receptors or transfecting agents, encapsulation in biopolymers (e.g., poly—1-4-N-acetylglucosamine polysaccharide; see, U.S. Pat. No. 5,635,493), encapsulation in liposomes, microparticles, or microcapsules; by administering it in linkage to a peptide or other ligand known to enter the nucleus; or by administering it in linkage to a ligand subject to receptor-mediated endocytosis (see, e.g., Wu and Wu, J. Biol. Chem., 1987, 62:4429-4432), etc. In another embodiment, a nucleic acid ligand complex can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Alternatively, the nucleic acid can be introduced intracellularly and incorporated within host cell DNA for expression by homologous recombination (Koller and Smithies, Proc. Natl. Acad. Sci. U.S.A., 1989, 86:8932-8935; Zijlstra, et al., Nature, 1989, 342:435-438). These methods are in addition to those discussed above in conjunction with “Viral and Non-viral Vectors”.

Alternatively, single chain antibodies can also be administered, for example, by expressing nucleotide sequences encoding single-chain antibodies within the target cell population by utilizing, for example, techniques such as those described in Marasco et al. Proc. Natl. Acad Sci. USA, 1993, 90:7889-7893).

The form and amount of therapeutic nucleic acid envisioned for use depends on the type of disease and the severity of the desired effect, patient state, etc., and can be determined by one skilled in the art.

### Formulations and Administration

Therapeutic compositions containing a construct for use in accordance with the present invention can be formulated in any conventional manner using one or more physiologically acceptable carriers or excipients.

Thus, the construct proteins or nucleic acids encoding them and their physiologically acceptable salts and solvents can be formulated for administration by inhalation (pulmonary) or insufflation (either through the mouth or the nose), by transdermal delivery, or by transmucosal administration, including, but not limited to, oral, buccal, nasal, ophthalmic, vaginal, or rectal administration.

For oral administration, the therapeutics can take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art. Liquid preparations for oral administration can take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate.

Preparations for oral administration can be suitably formulated to give controlled release of the active compound.

For buccal administration, the therapeutics can take the form of tablets or lozenges formulated in conventional manner.

For administration by inhalation, the therapeutics according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The therapeutics can be formulated for parenteral administration (e.g., intravenous, intramuscular, subcutaneous, intradermal) by injection, via, for example, bolus injection or continuous infusion. Formulations for injection can be presented in unit dosage form, e.g, in vials or ampoules or in multi-dose containers, with an added preservative. The compositions can take such forms as excipients, suspensions, solutions or emulsions in oily or aqueous vehicles, and can contain formulatory agents such as suspending, stabilizing and/or dispersing agents. Alternatively, the active ingredient can be in dry, lyophilized (i.e. freeze dried) powder form for constitution with a suitable vehicle, e.g., sterile pyrogen-free water or saline, before use.

The therapeutics can also be formulated in rectal compositions such as suppositories or retention enemas, e.g., containing conventional suppository bases such as cocoa butter or other glycerides.

In addition to the formulations described previously, the therapeutics can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

In a specific embodiment, the constructs can be delivered in poly-glycolic acid/lactic acid (PGLA) microspheres (see U.S. Pat. Nos. 5,814,344, 5,100,669, and 4,849,222; PCT Publication Nos. WO 95/11010 and WO 93/07861).

The constructs of the invention may be administered as separate compositions or as a single composition with more than one construct linked by conventional chemical or by molecular biological methods. Additionally, the diagnostic and therapeutic value of the constructs of the invention may be augmented by their use in combination with therapeutic agents used in the treatment of immune dysfunction.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water-free concentrate in a sealed container such as a vial or sachette indicating the quantity of active agent. Where the composition is administered by injection, a vial of sterile diluent can also be provided so that the ingredients may be mixed prior to administration.

The invention also provides a pharmaceutical pack or kit comprising one or more containers filled with one or more of the ingredients of the formulations of the invention. Associated with such container(s) can be a notice in the form prescribed by a governmental agency regulating the manufacture, use or sale of pharmaceuticals or biological products, which notice reflects approval by the agency of manufacture, use or sale for human administration.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. Composition comprising a compound of the invention formulated in a compatible pharmaceutical carrier may also be prepared, placed in an appropriate container, and labeled for treatment of an indicated condition.

Many methods may be used to introduce the vaccine formulations of the invention; these include but are not limited to oral, intracerebral, intradermal, intramuscular, intraperitoneal, intravenous, subcutaneous, intranasal routes, and via scarification (scratching through the top layers of skin, e.g., using a bifurcated needle) or any other standard routes of immunization.

### Effective Dose

The constructs and vectors described herein can be administered to a patient at therapeutically effective doses to treat certain inappropriate immune responses and immune dysfunctions, particularly autoimmune diseases. A therapeutically effective dose refers to that amount of a therapeutic sufficient to result in a healthful benefit in the treated subject.

The precise dose of the constructs to be employed in the formulation depends on the route of administration, and the nature of the patient's disease, and should be decided according to the judgment of the practitioner and each patient's circumstances according to standard clinical techniques. An effective dose is an amount effective to result in activation of the TPO receptor in vivo; preferably this dose induces megakaryocyte growth and differentiation. The term “induce” or “induction” means to increase by a measurable or observable amount. This induction of megakaryocyte growth and differentiation can mean an increase in megakaryocyte proliferation or increased expression of the platelet-specific antigen GPIIbIIIa, or both. The ability of a therapeutic composition of the inventions to produce this effect can be detected in vitro, e.g., by measuring the incorporation of labeled nucleotides (³H-thymidine) into the DNA of cells. Further experimental evidence of induction includes observing an increase in platelets in an animal model. The degree of induction is at least sufficient for measurement; preferably, it is at least about 5%; more preferably from about 5% to about 50%; more preferably still greater than about 50%; and most preferably greater than about 95%. Effective doses may be extrapolated from dose-response curves derived from animal model test systems, including transgenic animal models.

Toxicity and therapeutic efficacy of compounds can be determined by standard pharmaceutical procedures in cell culture or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Therapeutics that exhibit large therapeutic indices are preferred. While therapeutics that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage lies preferably within a range of circulating concentrations that include the ED₅₀ with little or no toxicity. The dosage can vary within this range depending upon the dosage form employed and the route of administration utilized. For any construct used in the method of the invention, the therapeutically effective dose can be estimated initially from cell culture assays. A dose can be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma can be measured, for example, by high performance liquid chromatography.

### EXAMPLES

The following Examples illustrate the invention without limiting its scope.

### Example 1

### Construction of the Variable Region Gene Containing the MPL Binding Sequence of Thrombopoietion

The monomer or dimer of MPL-binding peptide, IEGPTLRQWLAARA, identified by screening a peptide library and shown to be highly efficient in stimulating MPL-mediated thrombopoiesis (Cwirla et al., supra; U.S. Pat. No. 6,121,238) is inserted into one or more CDRs of a synthetic antibody using the standard “PCR knitting” procedure. The length of the peptide, 14 amino acids, is sufficiently short that it can be inserted into any of the six CDRs. Further, since it has already been shown that a pseudosymmetrical dimer of the peptide IEGPTLRQWLAARA (synthesized via the and-amino groups of a C-terminal, -alanine-modified lysine) is more potent than the peptide monomer (Cwirla et al., supra), molecular modeling could be used to determine which two CDRs would most likely mimic the structural conformation of the peptide dimer.

“PCR knitting”. In this protocol (see FIG. 2 and Bicknell and Vallee, Proc. Natl. Acad. Sci. USA 1989, 86:1537), positive and negative strand oligonucleotide primers are designed that overlap approximately ten residues at the 5′ end and contain novel sequences at their 5′ ends that encode the peptide sequences to be inserted. At the 3′ ends, the oligonucleotides contain sequences homologous to the framework sequences adjacent to the CDR being modified. Two polymerase chain reactions (PCR) are then performed using as primer pairs, one primer that encodes a portion of the peptide sequence and an appropriate primer up- or downstream from the CDR to be modified that corresponds to framework sequences that flank the CDR. The template DNA used for these two PCR reactions is the consensus variable region that has been described previously (FIGS. 1A and 1B) and was cloned into the shuttle vector pUC19 using standard techniques (Bicknell and Vallee, supra). PCR reactions are initiated by incubating the reactions for 5 minutes at 95° C. and then running 25 cycles of 30 seconds at 95° C, followed by 30 seconds at 55° C. and followed by 30 seconds at 72° C. After 25 cycles, an additional incubation is performed for 7 minutes at 72° C. The two PCR reactions produce DNA fragments that overlap by approximately 10 bp at one of their termini (FIG. 2; Table 1) and these fragments are purified using QIAquick PCR purification columns to the manufacturer's instructions (Qiagen).

The isolated fragments are then “knitted” together in another PCR reaction in which the flanking primers used in the first two PCR reactions described above are included in the reaction along with the two DNA fragments. As before, PCR reactions are begun by incubating for 5 minutes at 95° C., then 5 cycles were run of 30 seconds at 94° C., followed by 1 minute at 40° C. and followed by 30 seconds at 72° C. Twenty additional cycles were then performed of 30 seconds at 94° C., followed by 30 seconds at 55° C., and followed by 30 seconds at 72° C. After 25 cycles, an additional incubation is performed for 7 minutes at 72° C. The product of this reaction is a longer DNA fragment that results from the joining of the initial two fragments by selective annealing of the DNA fragments through the overlapping sequences present at one of their termini followed by amplification with the flanking primers (FIG. 2).

Table 1
Sequences of primers used for preparation of TPO
constructs.

| Primer | Sequence |
|---|---|
| TPOVHP2 | GCCACTGTCTCAGGGTGGGGCCCTCGATTGTGAATGTGTAGCCAGAAGC |
| TPOVHP3 | CCTGAGACAGTGGCTGGCCGCCAGAGCCTGGGTGAGGCAGGCTCCC |
| TPOVHP4 | GCCACTGTCTCAGGGTGGGGCCCTCGATGCCCATCCACTCCAGGCC |
| TPOVHP5 | CCTGAGACAGTGGCTGGCCGCCAGAGCCAGGGTTACTATAACTGCTGATAC |
| TPOVHP6 | GCCACTGTCTCAGGGTGGGGCCCTCGATCCTAGCGCAGTAGTAAACAG |
| TPOVHP7 | CCTGAGACAGTGGCTGGCCGCCAGAGCCTGGGGACAGGGAACACTG |
| TPOVLP2 | GCCACTGTCTCAGGGTGGGGCCCTCGATACATGTGATTGTCACCCGATC |
| TPOVLP3 | CCTGAGACAGTGGCTGGCCGCCAGAGCCTGGTATCAACAAAAGCCCGG |
| TPOVLP4 | GCCACTGTCTCAGGGTGGGGCCCTCGATATAGATCAACAACTTAGGAGCC |
| TPOVLP5 | CCTGAGACAGTGGCTGGCCGCCAGAGCCGGAGTGCCTAGTCGGTTC |
| TPQVLP6 | GCCACTGTCTCAGGGTGGGGCCCTCGATACAATAATAGGTAGCGAAGTC |
| TPOVLP7 | CCTGAGACAGTGGCTGGCCGCCAGAGCCTTCGGACAAGGAACCAAGG |
| Forward | GTAAAACGACGGCCAGT |
| Reverse | AGCGGATAACAATTTCACACAGGA |
| VLP3 | AGATCGGGTGACAATCACATG |
| VLP4 | CGTGTTCCACTTCCACTTCC |
| VLP5 | GGAGTGCCTAGTCGGTTC |
| VLP6 | CACCTTGGTTCCTTGTCCG |
| VHP3 | GTCTTGCAAGGCTTCTGGC |
| VHP4 | ATCAGCAGTTATAGTAACCCT |
| VHP5 | AGGGTTACTATAACTGCTGAT |
| VHIP6 | CCTAGCGCAGTAGTAAACAG |

To facilitate cloning of the modified CDR containing the MPL-binding sequence(s) back into the consensus variable region clone, unique restriction sites are inserted into the flanking sequences, one on either side of each CDR (FIG. 2), using the QuikChange™ kit from Stratagene according to the manufacturer's instructions. The “knitted” PCR fragment is then cleaved with the appropriate restriction enzymes (FIG. 2) and ligated into the cloned consensus variable region that has been cut with the same restriction enzymes.

The assembled, modified variable region containing the MPL-binding sequence(s) is then linked to the appropriate constant region clone. For assembly of the heavy chain of the antibody, a unique XhoI restriction enzyme site was engineered into both the 3′ end of the variable region and the 5′ end of the IgGI heavy chain constant region (Bicknell and Vallee, Proc. Natl. Acad. Sci. USA, 1988, 85:5961). At the 5′ end of the variable region, an EcoRI restriction site and a Kozak sequence are added using PCR. The modified heavy chain variable region is then joined to the heavy chain constant region by inserting the EcoRI/XhoI cut variable region fragment into a vector, obtained from Lonza Biologics PLC, containing the EcoRI/XhoI cut heavy chain constant region. For assembly of the light chain of the antibody, a unique BglII restriction enzyme site is engineered into the 3′ end of the light chain variable region and a BclI restriction enzyme site is added to the 5′ end of the light chain constant region (chain) (Bicknell and Vallee, 1988, supra). Similar to the heavy chain variable region, an EcoRI restriction site and Kozak sequence are added to the 5′ end of the light chain variable region using PCR. When BglII and BclI cut their respective cleavage sites, both enzymes leave overhangs with the same DNA sequence, which allows them to be ligated. Consequently, the modified light chain variable region clone is digested with EcoRI/BglII and the resulting fragment inserted into a second vector, obtained from Lonza Biologics PLC, containing the EcoRI/BclI cut light chain constant region.

In a final step, the heavy chain expression vector, containing the heavy chain variable region, and the light chain expression vector, containing the light chain variable region, are assembled into a single “double gene” expression vector. To assemble the “double gene” vector, the heavy chain expression vector is cleaved with BamHI and NotI. The resulting fragment contains the complete heavy chain expression cassette including the CMV promoter, the assembled heavy chain and a transcriptional terminator. The light chain expression vector is also cleaved with BamHI and NotI and after purifying the vector from a small fragment, the heavy chain expression cassette is inserted into the light chain vector.

Peptide sequences of variable regions containing MPL-TPO binding sequences. Inserted TPO sequences in each construct are indicated by underlining. Peptide sequences of consensus heavy chain (CONVH) and consensus light chain (CONVL) variable regions are also shown, with CDRs underlined.

TPOVHCDR1
MetAlaTrpValTrpThrLeuLeuPheLeuMetAlaAlaAlaGlnSerAlaGln
AlaGlnValGlnLeuValGlnSerGlyAlaGluValLysLysProGlyAlaSerValLysValSer
CysLysAlaSerGlyTyrThrPheThrIleGluGlyProThrLeuArgGlnTrpLeuAla
AlaArgAlaTrpValArgGlnAlaProGlyGlnGlyLeuGluTrpMetGlyTrpIleAsn
GlyAsnGlyAspThrAsnTyrAlaGlnLysPheGlnGlyArgValThrIleThrAlaAsp
ThrSerThrSerThrAlaTyrMetGluLeuSerSerLeuArgSerGluAspThrAlaVal
Tyr TyrCysAlaArgAlaProGlyTyrGlySerAspTyrTrpGlyGlnGlyThrLeuVal
ThrValSerSer
TPOVHCDR2
MetAlaTrpValTrpThrLeuLeuPheLeuMetAlaAlaAlaGlnSerAlaGln
AlaGlnValGlnLeuValGlnSerGlyAlaGluValLysLysProGlyAlaSerValLysValSer
CysLysAlaSerGlyTyrThrPheThrSerTyrAlaIleSerTrpAsnTrpValArgGln
AlaProGlyGlnGlyLeuGluTrpMetGlyIleGluGlyProThrLeuArgGlnTrpLeu
AlaAlaArgAlaArgValThrIleThrAlaAspThrSerThrSerThrAlaTyrMet Glu
LeuSerSerLeuArgSerGluAspThrAlaValTyrTyrCysAlaArgAlaProGlyTyr
GlySerAspTyrTrpGlyGlnGlyThrLeuValThrValSerSer
TPOVHCDR3
MetAlaTrpValTrpThrLeuLeuPheLeuMetAlaAlaAlaGlnSerAlaGln
AlaGlnValGlnLeuValGlnSerGlyAlaGluValLysLysProGlyAlaSeralLysValSer
CysLysAlaSerGlyTyrThrPheThrSerTyrAlaIleSerTrpAsnTrpValArgGln
AlaProGlyGlnGlyLeuGluTrpMetGlyTrpIleAsnGlyAsnGlyAspThrAsnTyr
AlaGlnLysPheGLnGlyArgValThrIleThrAlaAspThrSerThrSerThrAlaTyr
MetGluLeuSerSerLeuArgSerGluAspThrAlaValTyrTyrCysAlaArgIleGlu
GlyProThrLeuArgGlnTrpLeuAlaAlaArgAlaTrpGlyGlnGlyThrLeuValThrValSerSer
TPOVLCDR1
MetAlaTrpValTrpThrLeuLeuPheLeuMetAlaAlaAlaGlnSerAlaGlnAlaAsp
IleGlnMetThrGlnSerProSerSerLeuSerAlaSerValGlyAspArgValThrIleThr
CysIleGluGlyProThrLeuArgGlnTrpLeuAlaAlaArgAlaTrpTyrGlnGlnLys
ProGlyLysAlaProLysLeuLeuIleTyrAlaAlaSerSerLeuGluSerGlyValProSer
ArgPheSerGlySerGlySerGlyThrArgPheThrLeuThrIleSerSerLeuGlnPro
GluAspPheAlaThrTyrTyrCysGlnGlnTyrAsnSerLeuProTrpThrPheGlyGln
GlyThrLysValGluIle Lys
TPOVLCDR2
MetAlaTrpValTrpThrLeuLeuPheLeuMetAlaAlaAlaGlnSerAlaGlnAlaAsp
IleGlnMetThrGlnSerProSerSerLeuSerAlaSerValGlyAspArgValThrIleThr
CysArgAlaSerGlnSerIleSerAsnTyrLeuAlaTrpTyrGlnGlnLysProGlyLys
AlaProLysLeuLeuIleTyrIleGluGlyProThrLeuArnGlnTrpLeuAlaAlaArg
AlaGlyValProSerArgPheSerGlySerGlySerGlyThrArgPheThrLeuThrIle
SerSerLeuGlnProGluAspPheAlaThrTyrTyrCysGlnGlnTyrAsnSerLeuPro
TrpThrPheGlyGlnGlyThr LysValGluIleLys
TPOVLCDR3
MetAlaTrpValTrpThrLeuLeuPheLeuMetAlaAlaAlaGlnSerAlaGlnAlaAsp
IleGlnMetThrGlnSerProSerSerLeuSerAlaSerValGlyAspArgValThrIleThr
CysArgAlaSerGlnSerIleSerAsnTyrLeuAlaTrpTyrGlnGlnLysProGlyLys
AlaProLysLeuLeuIleTyrAlaAlaSerSerLeuGluSerGlyValProSerArgPheSer
GlySerGlySerGlyThrArgPheThrLeuThrIleSerSerLeuGlnProGluAspPhe
AlaThrTyrTyrCysIleGluGlyProThrLeuArgGlnTrpLeuAlaAlaArgAlaPhe
GlyGlnGlyThrLysValGluIleLys

The nucleotide sequences of the VH and VL consensus sequences are also provided:

VHCON
ATGGCTTGGGTGTGGACCTTGCTATTCCTGATGGCAGCTGCCCA
AAGTGCCCAAGCACAGGTTCAGCTGGTGCAGTCTGGCGCTGAGGTGA
AGAAGCCTGGCGCTTCTGTGAAGGTGTCTTGCAAGGCTTCTGGCTACA
CATTCACATCTTACGCTATATCTTGGAATTGGGTGAGGCAGGCTCCCG
GGCAGGGCCTGGAGTGGATGGGCTGGATAAATGGAAATGGAGATACA
AATTACGCCCAGAAGTTCCAGGGAAGGGTTACTATAACTGCTGATACT
TCTACTTCTACTGCTTACATGGAGCTCTCTTCTCTGAGGTCTGAGGATA
CTGCTGTTTACTACTGCGCTAGGGCTCCTGGCTACGGCTCTGATTATTG
GGGACAGGGAACACTGGTTACAGTCTCGAG
VLCON
ATGGCTTGGGTGTGGACCTTGCTATTCCTGATGGCAGCTGCCCA
AAGTGCCCAAGCAGATATCCAAATGACACAAAGTCCTAGTAGTTTGAG
TGCTAGTGTGGGAGATCGGGTGACAATCACATGTCGGGCTAGTCAAAG
TATCAGTAACTATTTGGCTTGGTATCAACAAAAGCCCGGGAAGGCTCC
TAAGTTGTTGATCTATGCTGCTAGTAGTTTGGAGAGTGGAGTGCCTAG
TCGGTTCAGTGGAAGTGGAAGTGGAACACGGTTCACCTTGACCATCAG
TAGTTTGCAACCTGAAGACTTCGCTACCTATTATTGTCAACAATATAAC
AGTTTGCCTTGGACCTTCGGACAAGGAACCAAGGTGGAGATCT

### Example 2

### Synthebody Expression and Purification

Once constructs are prepared, initial transfections are performed transiently in CHO-K1 cells. Co-transfections are performed using two single expression constructs (one encoding the light chain and another encoding the heavy chain of the immunoglobulin molecule) and a cationic liposomal reagent. Expression is measured at day 3 and day 7 by ELISA assay. The expressed antibody is purified using Protein-A or Protein-G column chromatography and characterized by HPLC and Western immunoblotting.

Prior to testing the ability of the synthebody to affect hematopoiesis in vivo (e.g., in model organisms), its activity is assessed in vitro by measuring (i) the ability to bind to the MPL receptor (using both direct binding studies and competition experiments) and (ii) activate MPL-mediated signaling cascades leading to changes in cell proliferation and/or differentiation and/or survival.

### Example 3

### Assessment of Synthebody Activity by Examination of Direct Binding to MPL Receptor

1×10⁷ KG-1 cells (Human acute myelogenous leukemia cell line) and 1×10⁷ TF-1 (Human bone marrow erythroleukemia cell line) cells were centrifuged and resuspended in 1.0 mL FACS buffer. 1×10⁶(100 μl) cells were transferred to eppendorf microcentrifuge tubes (1.5 mL) and spun at 3000 RPM for 1 min and buffer was aspirated from cell pellets. Synthebody binding was analyzed by adding 100 μl of 5, 50, 100, and 250 nM concentrations of TPO VLCDR2, TPO VHCDR3, and Human consensus, and 5, 50, and 250 nM concentrations of TPO VLCDR1 and TPO VHCDR1 synthebodies to cells. Synthebody binding was performed for 1 h at 4° C., followed by washing 2× with 1 ml of FACS buffer. Cells were incubated with FITC-Goat anti-human IgG diluted 1:20 in FACS buffer (50 μl/sample) for 1 h at 4° C. Cells were washed 2× with 1.0 mL FACS buffer and resuspended in 400 μl FACS buffer. Cells were transferred to Falcon #2052 round bottom tubes and FACS analysis was performed using a Becton Dickinson FACSscan. Data was reported as the percentage of cells which stained positive.

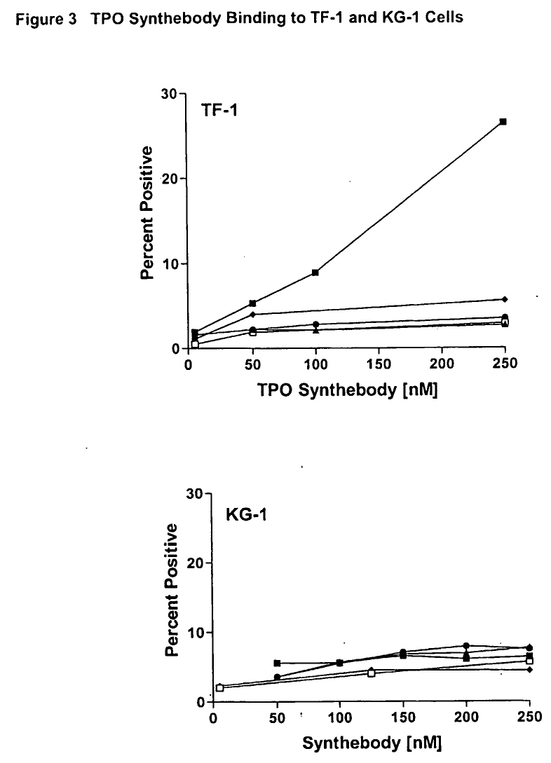

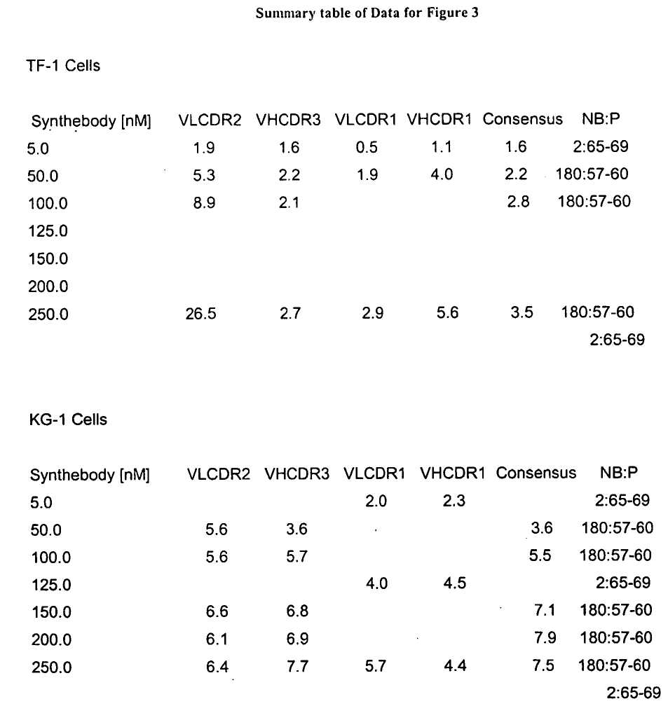

Synthebody binding was examined for TPO VLCDR2 (-▪-▪-▪-), TPO VHCDR3 (-▴-▴-▴-), TPO VLCDR1 (- [symbol] - [symbol] - [symbol] -), TPO VHCDR1 (-□-□-□-), and Human consensus (-●-●-●-) and the results of these binding studies to TF-1 and KG-1 cells are depicted in FIG. 3.

Without wishing to be bound by any theory, the data shown in FIG. 3 are consistent with the conclusion that the binding of the synthebody to the receptor is so tight that natural ligand could not compete for binding to the receptor with the synthebody.

In addition, several MPL agonist synthebody assays are conducted essentially as described in PCT Publication No. WO 99/10494.

CMK Assay for Induction of Platelet Antigen GPIIbIIIa Expression. CMK cells are maintained in RMPI 1640 medium (Sigma) supplemented with 10% fetal bovine serum and 10 mM glutamine. In preparation for the assay, the cells are harvested, washed and resuspended in serum-free GIF medium supplemented with 5 mg/l bovine insulin, 10 mg/l apo-transferrin, 1× trace elements. In a 96-well flat-bottom plate, the TPO standard or experimental agonist antibody samples are added to each well at appropriate dilutions in 100 μl volumes. 100 μl of the CMK cell suspension is added to each well and the plates are incubated at 37?C, in a 5% CO₂ incubator for 48 hours. After incubation, the plates are spun at 1000 rpm at 41 C for 5 minutes. Supernatants are discarded and 100 μl of the FITC-conjugated GPIIbIIIa 2132 mAb is added to each well. Following incubation at 4?C for 1 hour, plates are spun again at 1000 rpm for 5 minutes. The supernatants containing unbound antibody are discarded and 200 μl of 0.1% BSA-PBS wash is added to each well. The 0.1% BSA-PBS wash step is repeated three times. Cells are then analyzed on a FASCAN using standard one parameter analysis measuring relative fluorescence intensity.

KIRA ELISA for Measuring Phosphorylation of the MPL-Rse.gD Chimeric Receptor. This assay is used to determine whether the synthebodies of the invention activate the MPL receptor to a degree similar to that of cognate ligand (i.e., full-length TPO). A cDNA encoding chimeric MPL-Rse.gD receptor comprising the extracellular domain (ECD) of the MPL receptor and the transmembrane and intracellular domain (ICD) of Rse (Mark et al., J of Biol. Chem., 269: 10720-10728, 1994; Vigon et al., 1992, supra) with a C-terminal FLAG polypeptide is synthesized using PCR, inserted into the expression vector, and transfected into the host cell. Receptor phosphorylation upon addition of recombinant TPO (MPL agonist peptides or agonist synthebodies) is measured by KIRA ELISA using anti-phosphotyrosine 4G10 mAb (UBI, Lake Placid, N.Y.) biotinylated using long-arm biotin-N-hydroxysuccinamide (Biotin-X-NHS, Research Organics, Cleveland, Ohio).

TPO receptor-binding inhibition assay. NUNC 96-well immunoplates are coated with 50 μl of rabbit anti-human IgG Fc (Jackson Labs) at 2 μg/ml in carbonate buffer (pH 9.6) overnight at 4?C. After blocking with ELISA buffer (PBS, 1% BSA, 0.2% TWEEN 20), the plates are incubated for 2 hours with conditioned media from MPL-Ig-transfected 293 cells. Plates are washed, and 2.5 ng/ml biotinylated TPO is added in the presence or absence of various concentrations of antibodies. After incubation for 1 hour and washing, the amount of TPO bound is detected by incubation with streptavidin-HRP (Sigma) followed by TM13 peroxidase substrate (Kirkegaard & Perry). All dilutions are performed in ELISA buffer, and all incubations are at room temperature. Color development is quenched with H₃PO₄ and the absorbance is read at 450-650 nm.

HU-03 cell proliferation assay. GM-CSF-dependent cell line HU-01 derived from a patient with acute megakaryoblastic leukemia is obtained from Dr. D. Morgan, Halmemann University. The HU-03 cell line is derived from HU-01 cell line by adaptation to growth in the presence of rhTPO. HU-03 cells are maintained in RPMI 1640 media supplemented with 2% heat-inactivated human male serum and 5 ng/ml rhTPO. Before assay, cells are starved by removing TPO, decreasing serum concentrration to 1%, and adjusting the concentration of cells to 2.5×10⁵ cells/ml, followed by incubation for 16 hours. Cells are then washed and seeded into 96-well plates at a density of 5×10⁴ cells per well in medium containing TPO or antibodies at various concentrations. Quadruplicate assays are performed. 1 μCi ³H-thymidine is added to each well before incubation for 24 hours. Cells are collected with a Packard cell harvester and incorporation of ³H-thymidine is measured with a Top Count Counter (Packard).

TPO-synthebody competitive binding assays for HU-03 cells and human platelets. HU-03 cells are cultured as described above. Platelet rich plasma (PRP) is prepared by centrifugation of citrated whole blood at 400 g for 5 minutes. Binding studies are conducted within 3 hours of collection. Labeled TPO is prepared by indirect iodinationto specific activity of 15-50 μCi/μg protein (see Fielder et al., Blood, 89: 2782-2788, 1997).

100 pM iodinated TPO and either 2×10⁶ HU-03 cells in Hank's Balanced Salt Solution supplemented with 5 mg/ml bovine serum albumin (HBSSB), or 4×10⁷ platelets in plasma, are combined in a volume of 110 μl and incubated at 37?C for 30 minutes with varying concentrations of antibody in triplicate. HU-03 cells are agitated during the incubation period to keep them in suspension. The reaction mixture is overlayed on 1 ml 20% sucrose-HBSSB and microcentriftiged at 13,500 rpm for 5 minutes. The supernatants are aspirated, tube bottoms containing the cell pellets are cut off, and cell- or platelet-associated radioactivity is measured with an Iso Data Model 120 gamma counter.

Affinity determinations. The receptor-binding affinities of synthebodies are calculated from association and dissociation rate constants measured using a BIACORE surface plasmon resonance system (Pharmacia Biosensor). A biosensor chip is activated for covalent coupling of recombinant MPL receptor using N-ethyl-N′″-(3-dimethylaminopropyl)-carbodiimide hydrochloride (EDC) and N-hydroxysuccinimide (NHS) according to the supplier's (Pharmacia Biosensor) instructions. MPL is buffer-exchanged into 10 mM sodium acetate buffer (pH 4.5) and diluted to approximately 30 μg/ml. An aliquot (35 μl) is injected at a flow rate of 1 μl/min to achieve approximately 6400 response units (RU) of coupled protein. Finally, 1 M ethanolamine is injected as a blocking agent. For kinetics measurements, 1.5 serial dilutions of synthebody are injected in PBS/Tween buffer (0.05% Tween-20 in phosphate buffered saline) at 250?C using a flow rate of 20 μl/min. Equilibrium dissociation constants (Kd) from SPR measurements are calculated as k_{off}/kₒₙ. Standard deviations, sₒₙ for kₒₙ and s_{off} for k_{off}, are obtained from measurements with >4 protein concentrations (kₒₙ) or with >7 protein concentrations (k_{off}). Dissociation data are fit to a simple AB?A+B model to obtain k_{off}±s.d. (standard deviation of measurements). Pseudo-first order rate constant (ks) is calculated for each association curve, and plotted as a function of protein concentration to obtain kₒₙ±s.e. (standard error of fit). The resulting errors e[K] in calculated Kd's are estimated according to the following formula for propagation of errors: e[K]=[(kₒₙ)⁻²(S_{off})²+(k_{off})²(kₒₙ)⁻⁴(son)²]^{1/2} where s_{off} and sₒₙ are the standard errors in kₒₙ and k_{off}, respectively.

### Example 4

### Assessment of Synthebody Activity on Cellular Function

Several MPL agonist synthebody assays are conducted essentially as described in PCT Publication No. WO 99/10494.

Liquid suspension megakaryocytopoeisis assay. The effect of MPL agonist antibodies on human megakaryocytopoiesis is determined using a modification of the liquid suspension assay previously described (Grant et al., Blood 69:1334-1339, 1997). Buffy coats are collected from human umbilical cord blood and cells washed in phosphate-buffered saline (PBS) by centrifugation at 120 g for 15 minutes at room temperature to remove platelet-rich plasma. Cell pellets are resuspended in Iscove's modified Dulbecco's medium (IMDM, GIBCO) (supplemented with 100 units per ml penicillin and streptomycin), layered onto 60% percoll (density=1.077 gm/ml, Pharmacia), and centrifuged at 800 g for 20 minutes at room temperature. The light density mononuclear cells are collected from the interface and washed twice with IMDM. Cells are seeded at 1×10⁶ cells per ml in IMDM supplemented with 30% fetal bovine serum (FBS), 100 units per ml penicillin and streptomycin, and 20 μM 2-mercaptoethanol, into 24-well tissue culture plates (COSTAR). Serial dilutions of thrombopoietin (TPO), MPL agonist peptide or the synthebody are added to quadruplicate wells; with control wells containing no additional supplements. Final volumes are 1 ml per well. The cultures are grown in a humidified incubator at 37?C in 5% CO₂ for 14 days.

Megakaryocytopoiesis is quantified using radiolabeled murine mAb HP1-1D (provided by W. L. Nichols, Mayo Clinic) which has been shown to be specific for the human megakaryocyte glycoprotein GPIIbIIIa (Grant et al., supra). Cells are harvested from the tissue culture plates, washed twice with assay buffer (20% FBS, 0.002% EDTA in PBS), and resuspended in 100 μl assay buffer containing 20 ng iodinated HP1-1D (approximately 100,000 cpm). After incubation at room temperature for 1 hour, the cells are washed twice with assay buffer and the cell pellets counted with a gamma counter.

Effect of Synthebody on human bone marrow cells. CD34+ cells ARE isolated from human bone marrow, and megakaryocyte progenitors are assayed using MegaCult base medium kit (Stem cell Technologies). Cultures of CD34+ cells in serum-free semisolid agarose medium containing either rhTPO (R&D Systems) or synthebody are allowed to incubate at 37?C for 11 days. The number of megakaryocyte colonies containing three or more cells larger than 25 μm are counted by light microscopy.

Effect of synthebody on thrombopoiesis in mice. Subcutaneous injections of synthebody in mice are performed and platelet counts determined at designated times after the antibody administration. In addition, a histological examination of the bone marrow and spleen of these animals is performed to monitor an increase in the numbers of megakaryocytes.

Differentiation assay. Primary cultures of mouse bone marrow cells are used to determine if synthebody stimulates their differentiation. The differentiation is analyzed by measuring the levels of acetylcholinesterase (AchE, a marker enzyme of rodent megakaryocyte lineage cells

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, publications, test methods, literature, and other materials cited herein are hereby incorporated by reference.

What is claimed is:

1. A variant of an immnunoglobulin variable domain, said immunoglobulin variable domain comprising (A) at least one CDR region and (B) framework regions flanking said CDR, said variant comprising:
(a) said CDR region having added or substituted therein at least one binding sequence and
(b) said flanking framework regions,
wherein said binding sequence is heterologous to said CDR and is an antigenic sequence from a thrombopoietin receptor binding sequence.

2. The variant as define in claim 1, wherein the variable domain lacks an intrachain disulfide bond.

3. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii).

4. A variant as defined in claim 1, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

5. A variant as defined in claim 1, wherein (i) one or more amino acid residues in one or more of said flanking framework regions has been substituted or deleted, (ii) one or more amino acid residues has been added in one or more of said flanking framework regions, or (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

6. A variant of an immunoglobulin variable domain, said immunoglobulin variable domain comprising (A) at least one CDR region and (B) framework regions flanking said CDR, said variant comprising:
(a) said CDR region having added or substituted therein at least one amino acid sequence which is heterologous to said CDR and
(b) said flanking framework regions,
wherein said heterologous sequence is an antigenic sequence from a thrombopoietin receptor binding sequence.

7. A variant as defined in claim 6, wherein the variable domain lacks an intrachain disulfide bond.

8. A variant as defined in claim 6, wherein (i) one or more amino acid residues in one or more of said flanking framework regions has been substituted or deleted, (ii) one or more amino acid residues has been added in on or more of said flanking framework regions, or (iii) a combination of (i) and (ii).

9. A variant as defined in claim 6, wherein (i) one or more amino acid residues in one or more framework regions other than said framework regions flanking said CDR has been substituted or deleted, (ii) one or more amino acid residues has been added in one or more framework regions other than said framework regions flanking said CDR, or (iii) a combination of (i) and (ii).

10. A variant as defined in claim 6, wherein (i) one or more amino acid residues in one or more of said flanking framework regions has been substituted or deleted, (ii) one or more amino acid residues has been added in one or more of said flanking framework regions, (iii) a combination of (i) and (ii); and wherein (iv) one or more amino acid residues in one or more framework regions other than said framework regions flanking said CDR has been substituted or deleted, (v) one or more amino acid residues has been added in one or more framework regions other than said framework regions flanking said CDR, or (vi) a combination of (iv) and (v).

11. A variant as defined in claim 6, wherein said CDR is more than one CDR.

12. A variant as defined in claim 6, wherein said heterologous sequence is a CDR of a heavy chain variable region.

13. A variant as defined in claim 6, wherein said heterologous sequence is a CDR of a light chain variable region.

14. A variant as defined in claim 6, wherein said antigenic sequence is IEGPTLRQWLAARA.

15. A variant as defined in claim 6, which is an antibody.

16. A molecule comprising a variant as defined in claim 6.

17. A molecule comprising a variant as defined in claim 7.

18. A molecule comprising a variant as defined in claim 8.

19. A molecule comprising a variant as defined in claim 9.

20. A molecule comprising a variant as defined in claim 10.

21. A molecule comprising a variant as defined in claim 14.

22. A molecule as defined in claim 16, further comprising one or more constant domains from an immunoglobulin.

23. A molecule as defined in claim 16, further comprising a second variable domain linked to said variant.

24. A molecule as defined in claim 16, further comprising a second variable domain linked to said variant, and one or more constant domains from an immunoglobulin.

25. A molecule as defined in claim 16, wherein said CDR region is CDR 1.

26. A molecule as defined in claim 16, wherein said CDR region is CDR 2.

27. A molecule as defined in claim 16, wherein said CDR region is CDR 3.

28. A molecule as defined in claim 16, which is an antibody.

29. A molecule as defined in claim 16, which is derived from a human antibody.

30. A molecule as defined in claim 16, which is derived from a chimeric or a humanized antibody.

31. An immunoglobulin comprising a heavy chain and a light chain, wherein said heavy chain comprises a variant as defined in claim 6 and three constant domains from an immunoglobulin heavy chain, and said light chain comprises a second variable domain associated with said variant and a constant domain from an immunoglobulin light chain.

32. An immunoglobulin comprising a heavy chain and a light chain, wherein said light chain comprises a variant as defined in claim 6 and a constant domain from an immunoglobulin light chain, and said heavy chain comprises a second variable domain associated with said variant and three constant domains from an immunoglobulin heavy chain.

33. An isolated nucleic acid encoding a variant as defined in claim 1.

34. An isolated nucleic acid encoding a variant as defined in claim 6.

35. An isolated nucleic acid encoding a molecule as defined in claim 16.

36. An isolated nucleic acid encoding an immunoglobulin as defined in claim 29.

37. An isolated nucleic acid encoding an immunoglobulin as defined in claim 30.

38. A cell containing nucleic acid as defined in claim 31.

39. A cell containing nucleic acid as defined in claim 32.

40. A cell containing nucleic acid as defined in claim 33.

41. A cell containing nucleic acid as defined in claim 34.

42. A cell containing nucleic acid as defined in claim 35.

43. A recombinant non-human host containing nucleic acid as defined in claim 31.

44. A recombinant non-human host containing nucleic acid as defined in claim 32.

45. A recombinant non-human host containing nucleic acid as defined in claim 33.

46. A recombinant non-human host containing nucleic acid as defined in claim 34.

47. A recombinant non-human host containing nucleic acid as defined in claim 35.

48. A vaccine composition comprising a therapeutically or prophylactically effective amount of a variant as defined in claim 1, and an adjuvant.

49. A vaccine composition comprising a therapeutically or prophylactically effective amount of a variant as defined in claim 6, and an adjuvant.

50. A vaccine composition comprising a therapeutically or prophylactically effective amount of a variant as defined in claim 16, and an adjuvant.

51. A vaccine composition comprising a therapeutically or prophylactically effective amount of an immunoglobulin as defined in claim 29, and an adjuvant.

52. A vaccine composition comprising a therapeutically or prophylactically effective amount of an immunoglobulin as defined in claim 30, and an adjuvant.

53. A method of treating or preventing thrombocytopenia in a subject in need of such treatment or prevention, said method comprising administering to said subject a disease treating or preventing effective amount of a variant as defined in claim 1.

54. A method of treating or preventing thrombocytopenia in a subject in need of such treatment or prevention, said method comprising administering to said subject a disease treating or preventing effective amount of a variant as defined in claim 6.

55. A method of treating or preventing thrombocytopenia in a subject in need of such treatment or prevention, said method comprising administering to said subject a disease treating or preventing effective amount of a molecule as defined in claim 16.

56. A method of treating or preventing thrombocytopenia in a subject in need of such treatment or prevention, said method comprising administering to said subject a disease treating or preventing effective amount of an immunoglobulin as defined in claim 29.

57. A method of treating or preventing thrombocytopenia in a subject in need of such treatment or prevention, said method comprising administering to said subject a disease treating or preventing effective amount of a nucleic acid as defined in claim 31.

58. A method of treating or preventing thrombocytopenia in a subject in need of such treatment or prevention, said method comprising administering to said subject a disease treating or preventing effective amount of a vaccine composition as defined in claim 46.

59. A method of treating or preventing thrombocytopenia in a subject in need of such treatment or prevention, said method comprising administering to said subject a disease treating or preventing effective amount of a vaccine as defined in claim 48.

60. A variant of an immunoglobulin variable domain, said immunoglobulin variable domain comprising at least one CDR region, said variant comprising said CDR region having added or substituted therein at least one antigenic sequence from a thrombopoietin receptor binding sequence, said at least one sequence being selected from the group consisting of (a) a binding sequence heterologous to said CDR; (b) a CTL-epitope sequence; (c) a T-helper cell sequence; (d) a B-helper cell sequence; and (e) combinations thereof, wherein said at least one sequence is heterologous to said CDR and the variable domain lacks an intrachain disulfide bond.

61. A variant as claimed in claim 60 wherein said variable region comprises (a) a CDR1 region having said CTL epitope sequence substituted or added therein; (b) a CDR2 region having said T-helper cell substituted or added therein; and (c) a CDR3 region having said binding sequence of B-helper cell sequence substituted or added therein.

62. A variant as claimed in claim 60 wherein said binding sequence is IEGPTLRQWLAARA.

63. A variant as claimed in claim 60 which is an antibody.

64. A molecule comprising a variant as claimed in claim 60.

65. A molecule as claimed in claim 64 further comprising one or more constant domains from an immunoglobulin.

66. A molecule as claimed in claim 64 further comprising a second variable domain linked to said variant.

67. A molecule as claimed in claim 64 further comprising a second variable domain linked to said variant and one or more constant domains from an immunoglobulin.

68. A molecule as claimed in claim 64 which is an antibody.

69. A molecule as claimed in claim 64 which is derived from a human antibody.

70. A molecule as claimed in claim 64 which is derived from a chimeric or humanized antibody.

71. An immunoglobulin comprising a heavy chain and a light chain, wherein said heavy chain comprises a variant as claimed in claim 60 and three constant domains from an immunoglobulin heavy chain, and said light chain comprises a second variable domain associated with said variant and a constant domain from an immunoglobulin light chain.

72. An immunoglobulin comprising a heavy chain and a light chain, wherein said light chain comprises a variant as claimed in claim 60 and a constant domain from an immunoglobulin light chain, and said heavy chain comprises a second variable domain associated with said variant and three constant domains from an immunoglobulin heavy chain.

73. An isolated nucleic acid encoding a variant as claimed in claim 60.

74. An isolated nucleic acid encoding a molecule as claimed in claim 64.

75. An isolated nucleic acid encoding an immunoglobulin as claimed in claim 71.

76. An isolated nucleic acid encoding an immunoglobulin as claimed in claim 72.

77. A cell containing nucleic acid as claimed in claim 73.

78. A cell containing nucleic acid as claimed in claim 74.

79. A cell containing nucleic acid as claimed in claim 75.

80. A cell containing nucleic acid as claimed in claim 76.

81. A recombinant non-human host containing nucleic acid as claimed in claim 73.

82. A recombinant non-human host containing nucleic acid as claimed in claim 74.

83. A recombinant non-human host containing nucleic acid as claimed in claim 75.

84. A recombinant non-human host containing nucleic acid as claimed in claim 76.

85. A vaccine composition comprising a therapeutically or prophylactically effective amount of a variant as claimed in claim 60 and an adjuvant.

86. A vaccine composition comprising a therapeutically or prophylactically effective amount of a molecule as claimed in claim 64 and an adjuvant.

87. A vaccine compostion comprising a therapeutically or prophylactically effective amount of an immunoglobulin as claimed in claim 71 and an adjuvant.

88. A vaccine compostion comprising a therapeutically or prophylactically effective amount of an immunoglobulin as claimed in claim 72 and an adjuvant.

89. A method of treating or preventing thrombocytopenia in a subject in need of such treatment or prevention, said method comprising administering to said subject a disease treating or preventing effective amount of a variant as claimed in claim 60 and an adjuvant.

90. A method of treating or preventing thrombocytopenia in a subject in need of such treatment or prevention, said method comprising administering to said subject a disease treating or preventing effective amount of a molecule as claimed in claim 64 and an adjuvant.

91. A method of treating or preventing thrombocytopenia in a subject in need of such treatment or prevention, said method comprising administering to said subject a disease treating or preventing effective amount of an immunoglobulin as claimed in claim 71 and an adjuvant.

92. A method of treating or preventing thrombocytopenia in a subject in need of such treatment or prevention, said method comprising administering to said subject a disease treating or preventing effective amount of an immunoglobulin as claimed in claim 72 and an adjuvant.

93. A method of eliciting an anti-idiotypic response to an antigen in a subject in need of treatment or prevention of a disease condition associated with said antigen, said method comprising administering to said subject a disease treating or preventing effective amount of a variant as claimed in claim 60 and an adjuvant.

94. A method of eliciting an anti-idiotypic response to an antigen in a subject in need of treatment or prevention of a disease condition associated with said antigen, said method comprising administering to said subject a disease treating or preventing effective amount of a molecule as claimed in claim 64 and an adjuvant.

95. A method of eliciting an anti-idiotypic response to an antigen in a subject in need of treatment or prevention of a disease condition associated with said antigen, said method comprising administering to said subject a disease treating or preventing effective amount of an immunoglobulin as claimed in claim 71 and an adjuvant.

96. A method of eliciting an anti-idiotypic response to an antigen in a subject in need of treatment or prevention of a disease condition associated with said antigen, said method comprising administering to said subject a disease treating or preventing effective amount of an immunoglobulin as claimed in claim 72 and an adjuvant.
